SEQUENCE LISTING (9 sequences)

SEQ ID NO: 1 - DNA, 834 bp, Solanum tuberosum
  CDS (1)..(834)
atgagagctctctggaactccacctgcttgtccccagttcaaaataatccattgttattctctcgttcaagcaagaaatatgcgaattccttatgcaatttcaccaacaaatccttccagatttcttgcaaacttccagaaagtgaagttaaagagaaccatgctagatccagtagtaataagaagatggaggaatacaacttagctatgaagagaatgatgaggaatccttatgaatatcaccatgaacttggaatgaactacacattgataacagaagatctaattgttggctcccagcctcagaaaattgaagatatagattatttgaaggaagaggagaacgtagcttttatactaaacttgcagcaggacaaagatattgagttttggggaatagacctccagtctatcgttacaagatgttcagagcttggaattcatcacatgagaaggcctgcaagagattttgatccagattccctgaggagtgtattacctaaagctgtttcatcactggagtgggcgatttcagaaggaaaaggaagagtgtatgtacattgcactgctggattgggaagggcccctgctgtttcaattgcttatatgttctggttctgtgggatggatctaaatacagcttatgatacactcgtttcaaagagaccctgtgggcccaacaaaaggtcaatacagggagctacttatgatttggctaaaaatgatcagtggaaggagccctttgagaatctgccagattatgcctttgcggatgtagcagattgggagaggaaactgattcaagatcgtgtgcgagcccttcgtgacacttga

SEQ ID NO: 2 - protein, 277 aa, Solanum tuberosum
MRALWNSTCLSPVQNNPLLFSRSSKKYANSLCNFTNKSFQISCKLPESEVKENHARSSSNKKMEEYNLAMKRMMRNPYEYHHELGMNYTLITEDLIVGSQPQKIEDIDYLKEEENVAFILNLQQDKDIEFWGIDLQSIVTRCSELGIHHMRRPARDFDPDSLRSVLPKAVSSLEWAISEGKGRVYVHCTAGLGRAPAVSIAYMFWFCGMDLNTAYDTLVSKRPCGPNKRSIQGATYDLAKNDQWKEPFENLPDYAFADVADWERKLIQDRVRALRDT

SEQ ID NO: 3 - DNA, 885 bp, Solanum tuberosum
  CDS (1)..(885)
atggacttcgctagtatggaccgtgcacaactcactatggtgggatcagggttttctgctttgctttcaatgcatttcacaatacagctcttgtcacaacacctgttcttctggaaaaacccaaaggagcaaaaggcaataatcatgattatatgtatggctccactttatgccattgactcgtttgtgggtttgttagatattcgtggaagcaaaacatttttcatgtttctagactcagttaaagaatgctacgaggctgtggcaattgccaaatttttggctttgatgtatagtaatttgaatatatccatcagcaaaaacattgtgcctgatgaaatcaaggggagggaaattcatcattcctttccaatgactctatttcagccccgcactgctcgcttagatcaccggacactgaaacttctcaagcattggacatggcagtttgtcatcatccgtccagcatgctctatcttgatgatcacgttacagattcttgggttgtatccgagttggctcagctggacgtttaccatcattctcaatatttcattctcagtggccatgtactccttggttgttttctaccatgttttctcaaaggaactgcagccacacaaaccactttcaaagttcatctgcatcaaagggatagttttcttcagcttttggcaggggttgctggttaaaattctagtctcgtggggaattatcaaatctcaccatttttggttggatgtggagcaccttcaggaagccattcagaatgttttaatttgtgtggagatggttttcttttctgttatgcagcaatatgcataccatgtggctccttacagtggtgatgtcgaagcaaagttgaaactgaaaaaggatgactaa

SEQ ID NO: 4 - protein, 294 aa, Solanum tuberosum
MDFASMDRAQLTMVGSGFSALLSMHFTIQLLSQHLFFWKNPKEQKAIIMIICMAPLYAIDSFVGLLDIRGSKTFFMFLDSVKECYEAVAIAKFLALMYSNLNISISKNIVPDEIKGREIHHSFPMTLFQPRTARLDHRTLKLLKHWTWQFVIIRPACSILMITLQILGLYPSWLSWTFTIILNISFSVAMYSLVVFYHVFSKELQPHKPLSKFICIKGIVFFSFWQGLLVKILVSWGIIKSHHFWLDVEHLQEAIQNVLICVEMVFFSVMQQYAYHVAPYSGDVEAKLKLKKDD

SEQ ID NO: 5 - DNA, 2923 bp, Solanum tuberosum
  CDS (76)..(2445)
gaattcggcacgagatttttagggtttttggatgaggatttaagaggaaagatttgatttttttgaggattttggatggcgtcagctcaggtactaaaaaagcaagagcatttgcaagctgggaaaaagaagctagaggaatttcgtaagaagagagcagcagagaaggctaaaaagacaacttcaaatagtcaacagcttgcctctgatggaggcgttgataatcaacattcaggaaatgaacacactagaactaaggactccagtggagctgctacatctgatgctgttggcagatctgttctaaagccatctgaagtacatgctaagcatgattttgcgaagcctgatcttacccagaagtctgatttaatttttcctagtgatgctagtgctggtgctacgcctagtttgcacaaatactatgatgatgctgttgttaaagccaacagttatgattttggctcttccatctcagcattgtctcgtttagaaaataaagggtctagaagtgatgagaatcttaaggtttctcaaacagtaagtgatacttatgacaacactgggaagagggagagtgatggggccttagaaagtgttccatttgggtttgcgactaaccactctacagccacttttcctccattcctcaataatgacagaacttccagtcatttcacttatgatgatatgggtaaacgcatatcggaggagagccatgcaaaggatctttctgtaactaatgatggtacttctcatgcttttccggctaatgtatcaccttcaaatccattcgggtctcgtgacgacaaaccacgttatacagatcgctgggctagtgacatgacttccgcttcatatggtgattatgttcccggtgctaccacggacccacaattttatcctgaagttgggagaaatgttgctggtgtgggatctaataattttgtagtgcctgataaaggatacatccagttaaacagctctggtcttcactcaactaaaacttcttcttggacatcggactccaagtatgatggttttagttttgatgctagaagttcttctagttactcacagatgtctacactcacagctggggcgactgggaggagaactccatctttccttgattccatcaacatttcaaaagtctctgctgtatcccctccctcaataggatcagttactacagacacatatgactcaatggcttaccctagggacactctgggtttatcaaattctgagaacttgacaaattcttcaaaattttctggtaatgggtcagatctgtacaagcatgctgttgagaaagatatgggcaacttggacaacagacatccattttattcacagaagcaaaatgaagattttgctgcattagaacagcatattgaagatttaacacaagagaagttctctctacaacgtgctcttgaggcttcgcgaactctagcagagtctctagctgctgaaaattcaactctgacagatagttataatcaacagggaagttttgttggccaactaaaagctgagatggagaggttgcaagaggagattaaagcccatctgggtgagcttgaagctgtgaaaatggaatatgcaaatgtacaattggaatgtaatgctgctgatgagcgtgccaagttattggcttctgaagtgattggcttggaagagaaggcacttcgtctaagatctaatgagcttaaactggagaaagaattggagaagtcacaagctgaaatgtcttcttacaagaagaaaattgctagccttgaaaaagatcgtcaagatctgcaatcaacaatcgatgctttaaaggaagaaaagaagctcttgcaatccaaattcctaaaagcttctgctaatggaaagtcagttgatcctagcaggaatatgcctacaaaaatagatgtatcaacttctacagaggatcttcgtgaagataatatcgcaagtggtaccataaatgacactaatatggttggtattgatggccccaccacctcttccctacccgattttgggcagtttagtctcggaagtttgtcaccggccattcctccagatcagattaggatgatccaaaacattaatacattaatttctgagttagccttggagaaagacgaattaacaaaagccctgtcagttgaatcatctcagcgctctacattgaaggagttaaacagcgacttaactcggaagcttgaagttcaaacacaaagattggagcttttaactgctcaaagcatggcaaatgaaaacagccaagcaagacaaccagatgcagtgtctgtacatgacaatatgccatatgctgatgacaatatgcaatatgctgatgaaggagatgaggtggtggaacgggtattgggatggatcatgaaactatttcctggagggccatcaagacgacgaaccagcaagcttctttaatctccatggcaagagtacaatgatgagttatttctgcgaaaagctggtctttttctcctctccagccctgcatttttggtagccaaacggagtccggattttgtatattatccatggtttatacacacacattgcattcttttcccccagaagaagcaaattgtatctaagaggcattttggaagatggaattagctgttctcgacagttttggcaataaaacatgaccgtgttgtttacaagagaatccatttatcaaacgttgtaatgaagatcagatgtgtttagttgtcaatcatgagagaaaaaaaacttcaaatgtttcttacttttcatgtggaaatgatgtttagatcttttgacattgtattcgttatgttgtatttgttgaaagctggggcttaagaaaagcccaatatatcaaaaagatgattttggccgtcctttgtaaaaaaaaaaaaaaaaaaaactcgag

SEQ ID NO: 6 - protein, 790 aa, Solanum tuberosum
MASAQVLKKQEHLQAGKKKLEEFRKKRAAEKAKKTTSNSQQLASDGGVDNQHSGNEHTRTKDSSGAATSDAVGRSVLKPSEVHAKHDFAKPDLTQKSDLIFPSDASAGATPSLHKYYDDAVVKANSYDFGSSISALSRLENKGSRSDENLKVSQTVSDTYDNTGKRESDGALESVPFGFATNHSTATFPPFLNNDRTSSHFTYDDMGKRISEESHAKDLSVTNDGTSHAFPANVSPSNPFGSRDDKPRYTDRWASDMTSASYGDYVPGATTDPQFYPEVGRNVAGVGSNNFVVPDKGYIQLNSSGLHSTKTSSWTSDSKYDGFSFDARSSSSYSQMSTLTAGATGRRTPSFLDSINISKVSAVSPPSIGSVTTDTYDSMAYPRDTLGLSNSENLTNSSKFSGNGSDLYKHAVEKDMGNLDNRHPFYSQKQNEDFAALEQHIEDLTQEKFSLQRALEASRTLAESLAAENSTLTDSYNQQGSFVGQLKAEMERLQEEIKAHLGELEAVKMEYANVQLECNAADERAKLLASEVIGLEEKALRLRSNELKLEKELEKSQAEMSSYKKKIASLEKDRQDLQSTIDALKEEKKLLQSKFLKASANGKSVDPSRNMPTKIDVSTSTEDLREDNIASGTINDTNMVGIDGPTTSSLPDFGQFSLGSLSPAIPPDQIRMIQNINTLISELALEKDELTKALSVESSQRSTLKELNSDLTRKLEVQTQRLELLTAQSMANENSQARQPDAVSVHDNMPYADDNMQYADEGDEVVERVLGWIMKLFPGGPSRRRTSKLL

SEQ ID NO: 7 - DNA, 2102 bp, Solanum tuberosum
  CDS (65)..(1699)
ctgcaggcaaaaaaatacacattctgtttatatattttctattatcaaaaaacagaaataagaaatgactttaacacttcaatcatcagcttcttttatcaatttcaaagaaaccaaaggtgttaaagcacctgatgagttcttaggaatggtttcttttgcacaagccaagccatcatgccggctagtcgcgaaaagttcgatgcaagaagctcaactctcccatgagagaatcatggaagtgaagaaaattgagaaaagagagaagctacatgagttaccagctaatcacagcaatagaagtacaagggtacctgtttttgtgatgcttccacttgacaccatgactatgggagggaacttgaacaggccacgagcgatgaatgcgagtttgatggcgttgaaaagttctggagctgaaggggtgatggtggatgcttggtggggattggtggagaaagatggacctttgaagtataattgggaaggatatgctgagcttgtaaagatgtgtcaagaacatggattgaagcttcaagttgtcatgtcttttcatcagtgtggaggaaatgttggagattcttgcagtattcctctacctccatgggtacttgaagaaatcagcaagaatcctgaccttgtctacacagatagatcaggccggagaaatcctgagtatctatccttaggttgtgatatgttaccagtactcaaaggaagaacaccaattcaagtatacaccgactatatgaggagcttcagagaaagattcaacgaatacttgggaaacgtcatagtggaaatccaagtgggaatgggtccttgtggagagctaagatatccagcctatccagaaagcaatggtacatggaggtttcctggaattggagaattccaatgctatgacaagtacatgggagcttcattggcagcagtggccaaggcagctggaaaggatgactggggccagggagggcctcatgattctgggaagtacaaccagtttcctgaggatactggatttttccagagggatggaacatggaacagtgaatatggacagttcttcctagagtggtattcaggaaagctactggaacatggtgacagaatactagcagcaggagaaagtatataccaaggaactggggctaaactatctggaaaggtagctgggattcattggcattacaatactagatcacatgctgcagagttaacttcaggatattataatacaagacacagagatggttatctacctatagcacgtatgttagcgaaacatggtgctgtacttaactttacatgtatggaaatgagggatggtgaacagccccagagtgcaaactgttcaccagaaggcttagttcgacaagttaaaactgcagctagaactgctgaagtagaacttgctggagaaaatgctctagaaaggtatgatggaggagcattttctcaagttttggcaacaagcatgtcagattctggaaatggattgagtgcatttacattcttgcgaatgaacaaacggttgtttgagccagaaaattggcggaatctagtgcaatttgtgaagagcatgtctgaaggaggtcgaaatgctagccttccagagtgtgactcaagcaggacagacctctatgtaagatttatcaaagagagtcattctaagaaagctacagaggttgcagtagtgtaaagatacggaactgtatacatgtaatatagctatcccattgtaggttagcaaagaaaagtggcacatagagtactaaagtgtcatactcatagcacctagaagagtccacaagaatttgagcctgtgtctgaaattaaactatagaagacaagaaaaagaagctaagtacgccaatctttgccaccctggttcagaggttgtgaagctctgggtcattgggacagtgagaacaatgagacagtatcatacaaagatttggataagcatattcttttcattgtacaaaaattttaaaaaaaatctgccttccacatttattattgaagaataaaggagctaataatattgcttcctacagaggcattttcctgttaaaaaaaaaaaaaaaaaaaaactcgag

SEQ ID NO: 8 - protein, 545 aa, Solanum tuberosum
MTLTLQSSASFINFKETKGVKAPDEFLGMVSFAQAKPSCRLVAKSSMQEAQLSHERIMEVKKIEKREKLHELPANHSNRSTRVPVFVMLPLDTMTMGGNLNRPRAMNASLMALKSSGAEGVMVDAWWGLVEKDGPLKYNWEGYAELVKMCQEHGLKLQVVMSFHQCGGNVGDSCSIPLPPWVLEEISKNPDLVYTDRSGRRNPEYLSLGCDMLPVLKGRTPIQVYTDYMRSFRERFNEYLGNVIVEIQVGMGPCGELRYPAYPESNGTWRFPGIGEFQCYDKYMGASLAAVAKAAGKDDWGQGGPHDSGKYNQFPEDTGFFQRDGTWNSEYGQFFLEWYSGKLLEHGDRILAAGESIYQGTGAKLSGKVAGIHWHYNTRSHAAELTSGYYNTRHRDGYLPIARMLAKHGAVLNFTCMEMRDGEQPQSANCSPEGLVRQVKTAARTAEVELAGENALERYDGGAFSQVLATSMSDSGNGLSAFTFLRMNKRLFEPENWRNLVQFVKSMSEGGRNASLPECDSSRTDLYVRFIKESHSKKATEVAVV

SEQ ID NO: 9 - DNA, 32 bp, Artificial sequence
  Description of artificial sequence  PCR primer
gtccgcggatccatgactttaacacttcaatc

(19) United States
(12) Patent Application Publication
(10) Pub. No.: US 2006/0236426 A1
(43) Pub. Date: Oct. 19, 2006

(54) Nucleic acid molecules encoding starch degrading enzymes

(76) Inventors: Jens Kossmann, ROSKILDE (DK); Andreas Scheidig, Köln (DE); Anja Froehlich, Wustermark (DE)

Correspondence Address: BIRCH STEWART KOLASCH & BIRCH, PO BOX 747, FALLS CHURCH, VA, 22040-0747, US

(21) Appl. No.: 10/476,107

(22) PCT Filed: Apr. 25, 2002
(86) PCT No.: PCT/EP02/04609

§ 371 Date: Dec. 29, 2003

(30) Foreign Application Priority Data
Apr. 25, 2001 (EP) 01110005.4

(51) Int. Cl. A01H 5/00; C07H 21/04; C12N 9/32; C12N 15/82; C12N 5/04; A01H 1/00

(52) U.S. Cl. 800/284; 800/317.2; 536/23.2; 435/468; 435/419; 435/204

(57) ABSTRACT

Nucleic acid molecules encoding starch degrading enzymes are provided. Moreover, vectors, host cells and plant cells transformed by the herein-described nucleic acid molecules and plants containing them are provided. Furthermore, methods are described for preparing transgenic plants which show increased or reduced starch degradation.

The present invention relates to nucleic acid molecules encoding starch degrading enzymes. Moreover, this invention relates to vectors, host cells and plant cells transformed with the herein-described nucleic acid molecules, and plants containing said cells. Moreover, methods for preparing transgenic plants transformed with the described nucleic acid molecules are described.

Degradation of starch in plant organs is a process which influences in several aspects the usefulness of plant products. Depending on the plant species and on the type of organ it may be desirable to inhibit the degradation of starch or, to the contrary, to increase it. A reduction of starch degradation may, e.g., be desirable in fodder-plants, in particular in those which are conserved by silage or drying. An increase of the starch content would lead to a considerable increase of dry substance which would also broaden the C:N ratio thereby allowing to solve problems caused by a narrow C:N ratio. These problems include fermentation processes which may occur due to a too high pH value of plant organs caused by a narrow C:N ratio and which spoil the silage. Furthermore, also the gas accumulation in the rumen of ruminants, especially that which occurs in spring, is caused by a narrow C:N ratio of the grass which grows in this vegetation period and which is fed to the animals.

A reduction of starch may also be desirable in fruit. In a multitude of fruit starch is accumulated transiently and is mobilized during fruit ripening, i.e. converted into sugars. If it were possible to inhibit this process of starch degradation, it would be possible to increase the content of dry matter of the fruit. This would in particular be desirable in the case of tomato used for the production of ketchup because it would decrease the amount of energy which is otherwise necessary to evaporate exceeding water.

Also in connection with grapes a reduction of starch degradation could be desirable because an increased starch content would positively influence the sugar content of the grapes.

Furthermore, a reduction or inhibition of starch degradation could be desirable in potato tubers, in particular in connection with the so-called “cold-sweetening” which occurs during storage of tubers at low temperatures in order to suppress sprouting. The cold sweetening is due to a conversion of starch into glucose and fructose, the so-called reducing sugars, which lead to undesired browning reactions during frying processes.

Moreover, the reduction of starch degradation could also be desirable in connection with the generation of male sterile plants for the production of hybrid seed. In particular, it might be possible to produce male sterile plants by specifically inhibiting the starch degradation in pollen by repressing the responsible gene(s), since the pollen tube growth and the fertilization of the egg cell are energy dependent processes fueled in most plants by the degradation of starch.

On the other hand, there are situations in which it would be desirable to increase starch degradation in plant organs, e.g. when plant organs are used for the production of alcohol. However; the starch degradation in these cases should only occur during a certain time period, i.e. during the actual production process.

Thus, there is an interest to be able to modify starch degradation in certain plants or plant organs.

However, in order to be able to influence the degradation of starch in plants, in particular by genetic engineering, in a positive or negative manner it is necessary to have access to DNA sequences encoding proteins which play a role in starch degradation, in particular in those organs in which it is intended to modify starch degradation.

Therefore, the technical problem underlying the present invention is to provide nucleic acid molecules encoding proteins which are involved in starch degradation.

This problem is solved by the provision of the embodiments characterized in the claims.

Accordingly, the present invention relates to a nucleic acid molecule encoding a protein involved in starch degradation selected from then group consisting of
(a) nucleic acid molecules encoding at least the mature form of a protein which comprises the amino acid sequence indicated in SEQ ID NO: 2, 4, 6 or 8;
(b) nucleic acid molecules comprising the nucleotide sequence indicated in SEQ ID NO: 1, 3, 5 or 7 or a corresponding ribonucleotide sequence;
(c) nucleic acid molecules encoding a protein, the amino acid sequence of which has a homology of at least 40% to the amino acid sequence indicated in SEQ ID NO: 2, 4, 6 or 8;
(d) nucleic acid molecules, the complementary strand of which hybridizes with a nucleic acid molecule as defined in (a) or (b);
(e) nucleic acid molecules comprising a nucleotide sequence encoding a biologically active fragment of the protein which is encoded by a nucleic acid molecule as defined in any one of (a), (b), (c) or (d); and
(f) nucleic acid molecules, the nucleotide sequence of which deviates because of the degeneration of the genetic code from the sequence of a nucleic acid molecule as defined in any one of (b), (c), (d) or (e).

Consequently, the present invention relates to nucleic acid molecules encoding proteins involved in starch degradation, said molecules preferably encoding proteins comprising the amino acid sequence indicated in SEQ ID NO: 2, 4, 6 or 8.

The above-mentioned nucleic acid molecules SEQ ID NOs: 1, 3, 5 and 7 encode proteins which are involved in and strongly influence starch degradation. They were identified and isolated by using a functional screening assay employing E. coli strains accumulating linear α-1,4-glucans (see Example 1). With the help of these molecules it is now possible to modify the starch degradation (positively or negatively) in plant cells.

So far, sequences encoding enzymes which are involved in the degradation of starch or which initiate the degradation of starch nave not been described for most plant organs. The endosperm of cereal grains is the only plant system which is sufficiently understood in this regard. For all other plant organs it is so far not known which protein(s) is (are) responsible for starch degradation. The main reason for this is that the dominating starch hydrolysing activities in plant organs are localized in subcellular compartments in which no starch occurs. Starch is synthesized and degraded in the plastids. The products released by hydrolysis of the starch are then exported from the plastids for further use in the cell. In this regard the endosperm tissue is an exception since it looses its cellular and subcellular integrity during ripening. As a result also extraplastidial enzyme have access to starch granules and can degrade the starch.

The term “involved in starch degradation” means that the respective enzyme plays a role in the breakdown of starch. Such a breakdown may occur in different ways, e.g., by removal of glucose residues, maltose or maltooligosaccharides from the non-reducing or from the reducing end of a polysaccharide chain in the starch.

This removal may be achieved, e.g., by hydrolysis, i.e. the removed group is transferred to a water molecule (e.g. endohydrolases, exohydrolases), or by phosphorylysis, i.e. the removed group is transferred to a phosphate molecule (e.g. phosphorylases such as α-1,4 glucan phosphorylase which sets free glucose-1-phosphate molecules from the non-reducing end of a glucan chain).

Alternatively, the removal may be achieved by a reaction mechanism used by glucan lyases by which a glucose residue, preferably from the non-reducing end of a glucan chain, is converted into a 1,5-anhydro-fructose molecule which is set free.

Preferably, the term “involved in starch degradation” means a protein which can be identified in the functional assay described in Example 1 as having starch degrading activity. Such an assay in particular comprises the following steps:
(a) transforming a nucleic acid molecule encoding the protein into an E. coli strain which accumulates large amounts of α-1,4-glucans after growth on glucose containing medium, preferably into E. coli strain KV832 (Kiel et al., Molecular & General Genetics 207 (1987), 294-301) which is a mutant carrying an insertion in the glgB gene encoding glycogen branching enzyme and transformed with a plasmid conferring expression of an ADP-glucose pyrophosphorylase (PACAG; Koβmann et al., Planta 208 (1999), 503-511) with altered allosteric properties (Creuzat-Sigal et al. in: Biochemistry of the glycoside linkage, Ed.: Piras, Pontis; Academic Press, New York (1972), 647-680);
(b) plating and growing the transformed bacteria on glucose-containing medium;
(c) staining the bacterial colonies with iodine vapor;
(d) determining whether the bacterial colonies transformed with the nucleic acid molecule mentioned in step (a) show a lighter blue staining than untransformed control colonies, which show a dark blue staining with iodine vapor due to the presence of the linear α-1,4-glucans, or whether they show no staining at all, the lighter blue staining or lack of staining being indicative for the starch or glucan degrading activity of the protein.

The invention in particular relates to nucleic acid molecules containing the nucleotide sequence indicated under any one of SEQ ID NOs: 1, 3, 5 or 7 or a part thereof, and preferably to molecules, which comprise the coding region indicated in any one of SEQ ID NOs: 1, 3, 5 or 7 or corresponding ribonucleotide sequences.

Moreover, the present invention relates to nucleic acid molecules which encode a protein involved in starch degradation and the complementary strand of which hybridizes with one of the above-described molecules.

The present invention also relates to nucleic acid molecules which encode a protein, which has a homology, that is to say an identity of at least 40%, preferably at least 60%, preferably at least 70%, especially preferably at least 80% and in particular at least 90% to the entire amino acid sequence indicated in any one of SEQ ID NOs: 2, 4, 6 or 8, the protein being involved in starch degradation.

The present invention also relates to nucleic acid molecules, which encode a protein being involved in starch degradation and the sequence of which deviates on account of the degeneracy of the genetic code from the nucleotide sequences of the above-described nucleic acid molecules.

The invention also relates to nucleic acid molecules possessing a sequence which is complementary to the whole or a part of the above-mentioned sequences.

The nucleic acid sequence depicted in SEQ ID NO: 1 (also referred to as CSD12 herein) is a full-length cDNA sequence from potato with an open reading tram of 831 base pairs encoding a polypeptide of 277 amino acid residues. Computer analyses of the amino acid sequence (Emanuelsson et. al., Protein Science 8 (1999), 978-984; http://www.cbs.dtu.dk/services/ChloroP/) identify a plastidic transit peptide and indicate that the cleavage site between the transit peptide and the mature protein is between amino acid residues 56 and 57. Thus, the mature protein would comprise 221 amino acids. The predicted molecular weight of the unprocessed protein is 31.9 kDa and the predicted molecular weight of the mature protein is 25.5 kDa.

The nucleic acid sequence depicted in SEQ ID NO: 3 (also referred to as CSD23 herein) is a full-length cDNA clone from potato comprising an open reading frame of 882 nucleotides encoding a polypeptide of 294 amino acid residues. The predicted molecular weight of the polypeptide is 34.1 kDa.

Sequence comparisons revealed no significant homologies to known sequences available in data bases apart from an Arabidopsis EST without any identified function.

The nucleic acid sequence depicted in SEQ ID NO: 5 (also referred to as SHI herein) is a full-length cDNA clone from potato comprising an open reading frame of 2370 bp which encodes a polypeptide of 790 amino acid residues. The encoded protein has a predicted molecular weight of 86.6 kDa. It could be shown by expression of a chimeric protein containing the first 100 amino acid residues of the SHI protein and the GFP protein that a transit peptide for translocation into the plastids is present in the first 100 amino acids, since the chimeric protein is imported into the chloroplasts. The nucleotide sequence of SEQ ID NO: 5 shows some homology on the nucleotide sequence level to unidentified ESTs from tomato (AW 093761, AW 928571, AW 038351), cotton (AW 727818), aspen (AI 164445), soybean (AI 988543) and maize (AW 566133).

Transgenic potato plants containing antisense-constructs of SEQ ID NO: 5 can no longer mobilize transitory starch in their leaves which results in a so-called “starch-excess” phenotype. When expressed in E. coli, the SHI sequence (SEQ ID NO: 5) leads to a rapid degradation of amylopectin when amylopectin is solved and incubated with extracts from E. coli cells expressing SHI. Thin layer chromatography revealed that SHI activity leads to the release of maltooligosaccharides of different length, such as maltose, maltotriose and maltotetraose.

The nucleic acid sequence depicted in SEQ ID NO: 7 shows homology to a sequence encoding a plastidic β-amylase (Lao et al., Plant J. 20 (1999), 519-527). SEQ ID NO: 7 is a full-length cDNA clone comprising an open reading frame of 1635 bp which encodes a polypeptide of 545 amino acid residues. The predicted molecular weight is 61 kD. Although no plastidic transit peptide could be identified by computer analyses, import experiments with isolated chloroplasts from pea show that the protein is indeed imported into the chloroplast. Therefore, it is called potato plastidic targeted β-amylase (ppt-β-amylase).

Transgenic potato plants containing antisense constructs of SEQ ID NO: 7 show the so-called “starch-excess” phenotype, which means that they are no longer capable of mobilizing the transitory starch produced in their leaves.

Within the present invention the term “hybridization” means hybridization under conventional hybridization conditions (also referred to as “low stringency conditions”), preferably under stringent conditions (also referred to as “high stringency conditions”), as for instance described in Sambrook et al., Molecular Cloning, A Laboratory Manual, 2^{nd} edition (1989) Cold Spring Harbour Laboratory Press, Cold Spring Harbour, N.Y. Within an especially preferred meaning the term “hybridization” means that hybridization occurs under the following conditions:
Hybridization buffer: 2×SSC; 10× Denhardt solution (Fikoll 400+PEG+BSA; ratio 1:1:1); 0.1% SDS; 5 mM EDTA; 50 mM Na₂HPO₄; 250 μg/ml of herring sperm DNA; 50 μg/ml of tRNA; or
   0.25 M of sodium phosphate buffer, pH 7.2;
   1 mM EDTA
   7% SDS
Hybridization temperature T=60° C.
Washing buffer: 2×SSC; 0.1% SDS
Washing temperature T=60° C.

Nucleic acid molecules which hybridize with a nucleic acid molecule of the invention can, in principle, encode a protein involved in starch degradation from any organism expressing such proteins.

Nucleic acid molecules which hybridize with a molecule of the invention can for instance be isolated from genomic libraries or cDNA libraries of plants. Alternatively, they can be prepared by genetic engineering or chemical synthesis.

Such nucleic acid molecules may be identified and isolated with the use of a molecule of the invention or parts of such a molecule or reverse complements of such a molecule, for instance by hybridization according to standard methods (see for instance Sambrook et al., 1989, Molecular Cloning. A Laboratory Manual, 2^{nd} edition, Cold Spring Harbor Laboratory Press, Cold Spring Harbor, N.Y.).

Nucleic acid molecules possessing the same or substantially the same nucleotide sequence as indicated in SEQ ID NOs: 1, 3, 5 or 7 or parts thereof can, for instance, be used as hybridization probes. The fragments used as hybridization probes can also be synthetic fragments which are prepared by usual synthesis techniques, and the sequence of which substantially coincides with that of a nucleic acid molecule according to the invention.

The molecules hybridizing with a nucleic acid molecule of the invention also comprise fragments, derivatives and allelic variants of the above-described nucleic acid molecules encoding a protein involved in starch degradation. Herein, fragments are understood to mean parts of the nucleic acid molecules which are long enough to encode one of the described proteins, preferably being involved in starch degradation. In this connection, the term derivative means that the sequences of these molecules differ from the sequence of an above-described nucleic acid molecule in one or more positions and show a high degree of homology to such a sequence. In this context, homology means a sequence identity of at least 40%, in particular an identity of at least 60%, preferably of at least 65%, more preferably of at least 70%, even more preferably of at least 80%, in particular of at least 85%, furthermore preferred of at least 90% and particularly preferred of at least 95%. Most preferably homology means a sequence identity of at least n %, wherein n is an integer between 40 and 100, i.e. 40≦n≦100. Deviations from the above-described nucleic acid molecules may have been produced, e.g., by deletion, substitution, insertion and/or recombination.

Preferably, the degree of homology is determined by comparing the respective sequence with the nucleotide sequence of the coding region of SEQ ID No: 1, 3, 5 or 7. When the sequences which are compared do not have the same length, the degree of homology preferably refers to the percentage of nucleotide residues in the shorter sequence which are identical to nucleotide residues in the longer sequence. The degree of homology can be determined conventionally using known computer programs such as the ClustalW program (Thompson et al., Nucleic Acids Research 22 (1994), 4673-4680) distributed by Julie Thompson (Thompson@EMBL-Heidelberg.DE) and Toby Gibson (Gibson@EMBL-Heidelberg.DE) at the European Molecular Biology Laboratory, Meyerhofstrasse 1, D 69117 Heidelberg, Germany. ClustalW can also be downloaded from several websites including IGBMC (Institut de Génétique et de Biologie Moléculaire et Cellulaire, B. P. 163, 67404 Illkirch Cedex, France; ftp://ftp-igbmc.u-strasbg.fr/pub/) and EBI (ftp://ftp.ebi.ac.uk/pub/software/) and all sites with mirrors to the EBI (European Bioinformatics Institute, Wellcome Trust Genome Campus, Hinxton, Cambridge CB10 1SD, UK).

When using ClustalW program version 1.8 to determine whether a particular sequence is, for instance, 90% identical to a reference sequence according to the present invention, the settings are set in the following way for DNA sequence alignments:

KTUPLE=2, TOPDIAGS=4, PAIRGAP=5, DNAMATRIX:IUB, GAPOPEN=10, GAPEXT=5, MAXDIV=40, TRANSITIONS: unweighted.

For protein sequence alignments using ClustalW program version 1.8 the settings are the following: KTUPLE=1, TOPDIAG=5, WINDOW=5, PAIRGAP=3, GAPOPEN=10, GAPEXTEND=0.05, GAPDIST=8, MAXDIV=40, MATRIX=GONNET, ENDGAPS(OFF), NOPGAP, NOHGAP.

Furthermore, homology means preferably that the encoded protein displays a sequence identity of at least 40%, more preferably of at least 60%, even more prefer ably of at least 80%, in particular of at least 90% and particularly preferred of at least 95% to the amino acid sequence depicted under SEQ ID NO: 2, 4, 6 or 8. Most preferably homology means that there is a sequence identity of at least n %, wherein n is an integer between 40 and 100, i.e. 40≦n≦100.

With respect to SEQ ID NO: 1 (CSD12) homology preferably means that the encoded protein has a sequence identity of at least 62.5%, more preferably of at least 65%, even more preferably of at least 70% and particularly preferred of at least 95% to the amino acid sequence of SEQ ID NO: 2.

With respect to SEQ ID NO: 3 (CSD23) homology preferably means that the encoded protein has a sequence identity of at least 87%, more preferably of at least 90%, even more preferably of at least 95% and particularly preferred of at least 97% to the amino acid sequence of SEQ ID NO: 4.

With respect to SEQ ID NO: 5 (SHI) homology preferably means that the encoded protein has a sequence identity of at least 65%, more preferably of at least 75%, even more preferably of at least 86%, furthermore preferred of at least 95% and particularly preferred of at least 99% to the amino acid sequence of SEQ ID NO: 6. With respect to SEQ ID NO: 7 homology preferably means that the encoded protein has a sequence identity of at least 81%, more preferably of at least 85%, even more preferably of at least 95% and particularly preferred of at least 97% when compared to the sequence of SEQ ID NO: 8.

Homology, moreover, means that there is a functional and/or structural equivalence between the corresponding nucleic acid molecules or proteins encoded thereby. Nucleic acid molecules which are homologous to one of the above-described molecules and represent derivatives of these molecules are generally variations of these molecules which represent modifications having the same biological function. They may be either naturally occurring variations, for instance sequences from other microorganisms, or mutations, and said mutations may have formed naturally or may have been produced by deliberate mutagenesis. Furthermore, the variations may be synthetically produced sequences. The allelic variants may, e.g., be naturally occurring variants or synthetically produced variants or variants produced by recombinant DNA techniques.

The proteins encoded by the different variants of one of the nucleic acid molecules of the invention possess certain characteristics they have in common. These include for instance enzymatic activity, molecular weight, immunological reactivity, conformation, etc., and physical properties, such as for instance the migration behavior in gel electrophoreses, chromatographic behavior, sedimentation coefficients, solubility, spectroscopic properties, stability, pH optimum, temperature optimum etc.

One characteristic of the proteins encoded by one of the nucleic acid molecules of the invention is that they are involved in starch degradation. This activity can be assessed by the assay as described above.

Alternatively, starch degrading activity can be tested by separating the protein or a protein extract of a cell expressing the protein by polyacrylamid gel electrophoresis in amylose or amylopectin-containing gels under non-denaturing conditions and subsequently staining the gel in Lugol's solution. Proteins with starch degrading activity degrade the amylose and/or amylopectin present in the gel thereby leading to a lighter staining at their location in the gel.

Furthermore, starch degrading activity can be verified by incubating an amylose or amylopectin solution with an extract derived from cells expressing the protein to be tested and subsequently staining it with iodine. If the protein possesses no starch degrading activity the solution will show a violet staining. If the protein possesses starch degrading activity, either no or only a weak violet staining can be seen.

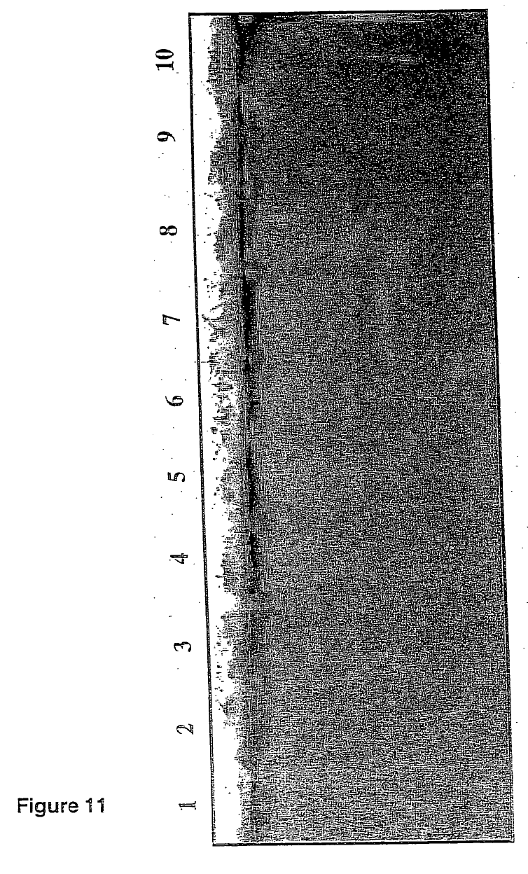

In the case of the protein encoded by SEQ ID NO: 3 (CSD23) or homologs thereof, the encoded protein has the property that it has hydrolytic activity. Furthermore, such a protein is characterized in that it only degrades linear, not-branched glucans but no amylopectin. This property can be tested for as described in Examples 3 and 7, e.g., by separating soluble protein fractions of cells expressing the protein in a discontinuous PAGE using as separating gels a gel which contains amylose and amypectin, respectively, and staining the gel with iodine. A protein encoded by SEQ ID NO: 3 or a homolog thereof can only degrade amylose but not amylopectin (see also FIG. 11) which is detectable by the negative staining of the gel.

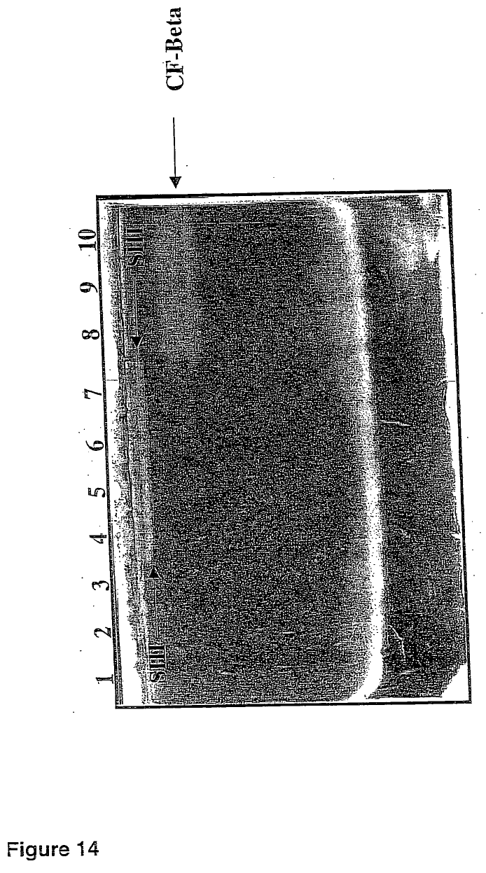

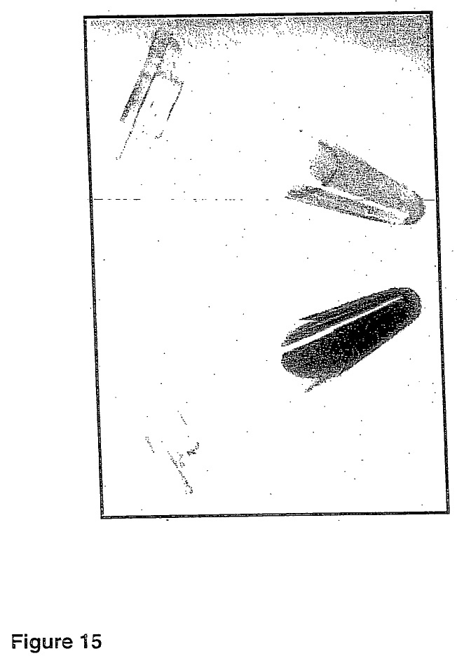

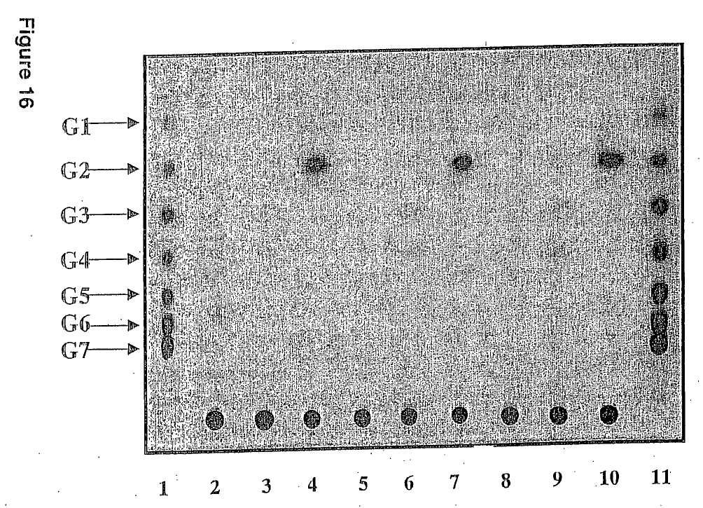

In the case of the protein encoded by SEQ ID NO: 5 (SHI) or homologs thereof, the encoded protein has the property that it has hydrolytic activity. This can be shown in discontinuous PAGE as described in Example 10 and FIG. 14. Such a protein is furthermore characterized by its capacity to degrade amylopectin (see FIGS. 14 and 15), and in particular solved amylopectin. This property can be easily tested for by incubating the protein with an amylopectin solution and subsequently staining it with iodine. The loss of blue staining is indicative for the degradation of amylopectin. Furthermore, an SHI protein has the property that its activity leads to the release of maltooligosaccharides, preferably of maltose, maltotriose and/or maltotetraose, from starch. This can be verified by incubating the protein with soluble starch and separating the products of starch degradation by thin layer chromatography (TLC) as described, e.g., in Examples 3 and 10 and as shown in FIG. 16.

An SHI protein preferably also has α-amylase activity. This activity can be tested for as described in the Examples, in particular in Examples 3 and 11. Preferably, it is tested by using p-nitrophenyl-maltoheptaose (PNPG7) which is blocked at the non-reducing end as a substrate and determining whether it is used as a substrate by the protein.

An SHI protein is furthermore preferably characterized by comprising a plastid targeting sequence and by its ability to be imported into chloroplasts, in particular isolated chloroplasts. The latter property can be tested for by doing import experiments as described, e.g., in Examples 4 and 12.

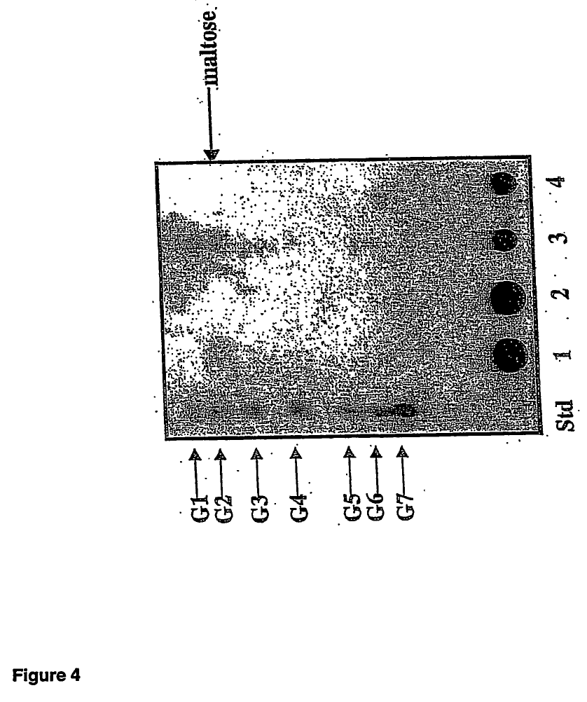

In the case of SEQ ID NO: 5, the encoded protein (SHI) preferably has a molecular weight of 80 to 90 kDa, preferably of 82 to 88 kDa, more preferably of 83 to 87 kDa and most preferably of about 86 kDa when calculated from the amino acid sequence. In the case of the protein encoded by SEQ ID NO: 7 (ppt-β-amylase) or homologs thereof, the encoded protein is characterized in that it has β-amylase activity. This activity can, e.g., be tested for as described in Example 3. Preferably it is determined by assessing whether the protein can degrade malto-oligosaccharides linked to a p-nitrophenyl group by a glucosidic bond at the reducing end. In particular, the specific substrate of β-amylase is non-blocked p-nitrophenyl-maltopentaose (PNPG5). β-amylase activity can also be tested by incubating the protein with solubilised starch or with raw potato starch granules and separating the reaction products by thin layer chromatography (TLC). The product of β-amylase activity is maltose only, while α-amylases, e.g., produce a series of malto-oligosaccharides (see Example 3 and FIG. 4).

Furthermore, a β-amylase protein according to the present invention is preferably characterized as comprising a plastid targeting sequence and by its ability to be imported into chloroplasts, in particular isolated chloroplasts. The latter property can be tested for by doing import experiments as described, e.g., in Example 4.

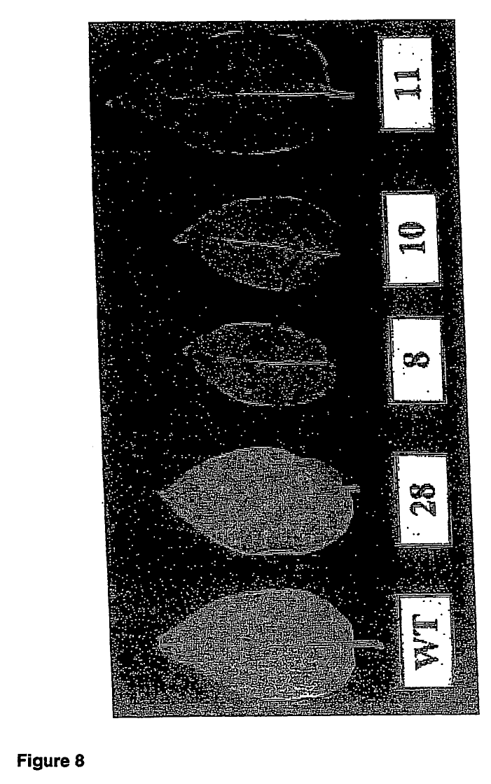

Moreover, the encoded proteins, in particular those encoded by SEQ ID NO: 5 and SEQ ID NO: 7, and their homologs show the characteristic property that plants in which their activity is reduced, e.g. via an antisense approach, show a so-called “starch excess” phenotype which means that they are no longer capable of mobilizing the starch synthesized in their leaves (transitory starch). This means that such plants show an accumulation of starch in their leaves. This property can be tested for, e.g., as described in Examples 5 and 13. In particular, source leaves of the plants are kept in darkness for different time intervals and then stained with iodine in order to determine their starch content. Leaves of plants which cannot mobilize the transitory starch in the dark show a blue staining, at least blue staining in these leaves is e.g. stronger or staining is apparent after longer time intervals in the dark as compared to staining that may occur in leaves of corresponding wild-type plants (see FIGS. 8, 19, 20 and 21).

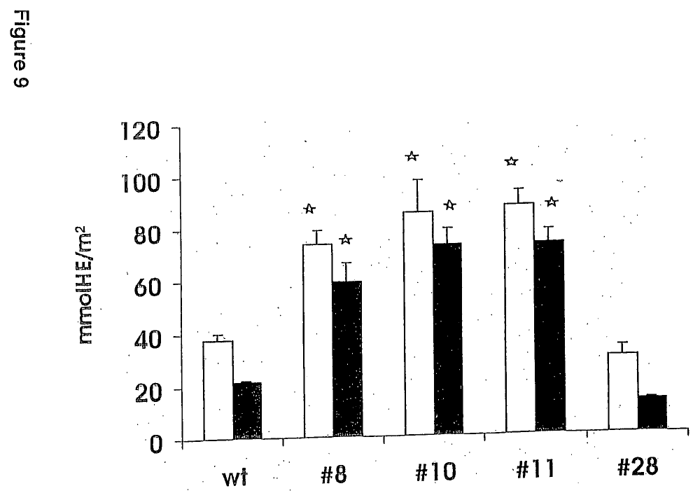

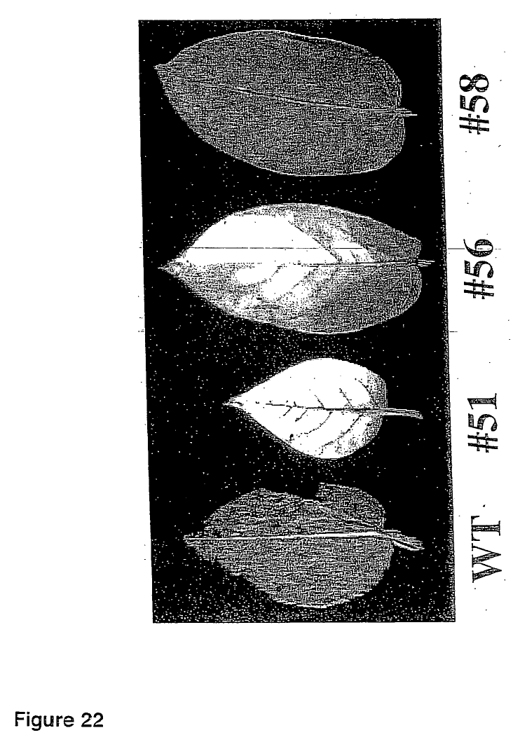

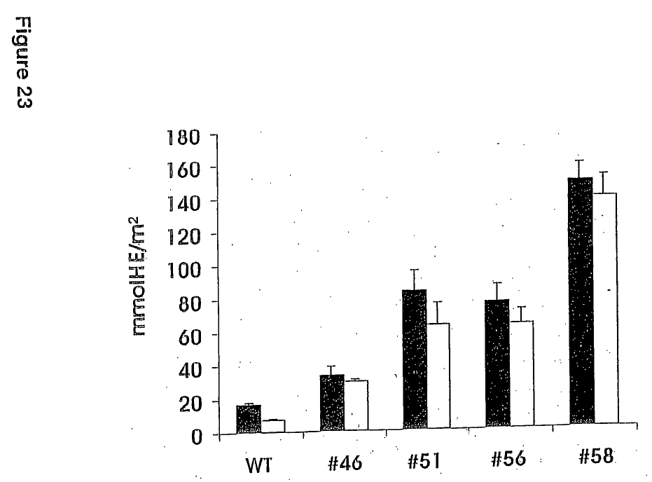

Furthermore, the accumulation of transitory starch in the leaves can also be tested for by enzymatically determining the starch content in the leaves. This can be done, e.g. as described in Müller-Röber et al. (EMBO J. 11 (1992), 1229-1238). Leaves of plants in which the activity of an SHI protein or a β-amylase according to the invention is reduced show preferably an increase in starch content of at least 50%, more preferably of at least 100%, even more preferably of at least 200%, still more preferably of at least 400% and, particularly preferred of at least 600% when compared to leaves of corresponding wild-type plants (see, e.g. FIGS. 9 and 23). Moreover, the leaves of plants which have a reduced activity of an SHI protein or a β-amylase according to the invention survive longer dark periods than corresponding leaves of corresponding wild-type plants (see FIG. 22).

The nucleic acid molecules of the invention can be DNA molecules, e.g. genomic DNA or cDNA. Moreover, the nucleic acid molecules of the invention may be RNA molecules. The nucleic acid molecules of the invention can be obtained for instance from natural sources or may be produced synthetically or by recombinant techniques.

The nucleic acid molecules of the invention allow host cells to be prepared which produce a protein involved in starch degradation of high purity and/or in sufficient quantities, and genetically engineered plants with a modified activity of these proteins. Within the framework of the present invention the term “high purity” means that a protein according to the invention displays a degree of purity of at least 80%, preferably of at least 90%, even more preferably of at least 95%.

In a preferred embodiment, the nucleic molecules of the invention are derived from plants, preferably from starch storing plants, more preferably from plants from the Solanaceae family and particularly preferred from Solanum tuberosum.

The invention also relates to oligonucleotides specifically hybridizing to a nucleic acid molecule of the invention. Such oligonucleotides have a length of preferably at least 10, in particular at least 15, and particularly preferably of at least 50 nucleotides. They are characterized in that they specifically hybridize to a nucleic acid molecule of the invention, that is to say that they do not or only to a very minor extent hybridize to nucleic acid sequences encoding other proteins, in particular other starch degrading enzymes. The oligonucleotides of the invention can be used for instance as primers for amplification techniques such as the PCR reaction or as a hybridization probe to isolate related genes.

Moreover, the invention relates to vectors, in particular plasmids, cosmids, viruses, bacteriophages and other vectors commonly used in gene technology, which contain one of the above-described nucleic acid molecules of the invention. In a preferred embodiment of the invention, the vectors of the invention are suitable for the transformation of plant cells. Particularly preferred, such vectors permit the integration of a nucleic acid molecule of the invention, possibly together with flanking regulatory regions, into the genome of the plant cell. Examples thereof are binary vectors which can be used in the Agrobacteria-mediated gene transfer, and some of which are already commercially available.

In another preferred embodiment, the nucleic acid molecule contained in the vectors is linked to regulatory elements ensuring transcription and synthesis of a translatable or non-translatable (e.g. antisense or ribozyme) RNA in prokaryotic or eukaryotic cells.

The expression of the nucleic acid molecules of the invention in prokaryotic or eukaryotic cells, for instance in Escherichia coli, is interesting because it permits a more precise characterization of the biological and/or enzymatic activities of the enzymes encoded by these molecules. Moreover, it is possible to express the proteins in such prokaryotic or eukaryotic cells which are free from interfering enzymes. In addition, it is possible to insert different mutations into the nucleic acid molecules by methods usual in molecular biology (see for instance Sambrook et al., 1989, Molecular Cloning, A Laboratory Manual, 2^{nd} edition, Cold Spring Harbor Laboratory Press, Cold Spring Harbor, N.Y.), leading to the synthesis of proteins possibly having modified biological properties. On the one hand it is possible in this connection to produce deletion mutants in which nucleic acid molecules are produced by progressive deletions from the 5′ or 3′ end of the coding DNA sequence, and said nucleic acid molecules lead to the synthesis of correspondingly shortened proteins. Such deletions at the 5′ end of the nucleotide sequence for instance allow amino acid sequences to be identified which are possibly present and which are responsible for the secretion of the protein or for the localization in the plastids, vacuole, mitochondria or the apoplast.

This permits the deliberate preparation of proteins which are no longer secreted by the removal of the corresponding sequences, but remain within the cell of the corresponding host organism or are localized in other compartments, for instance in the plastids, mitochondria, vacuole, on account of the addition of other signal sequences.

On the other hand, the introduction of point mutations is also conceivable at positions at which a modification of the amino acid sequence for instance influences the biological and/or enzymatic activity or the control of the protein/enzyme. In this manner, it is for instance possible to produce mutants which possess a modified stereo and regio selectivity or a modified Kₘ value or which are no longer subject to the control mechanisms normally existing in the cell and realized via an allosteric control or covalent modification.

Moreover, mutants possessing a modified substrate or product specificity can be prepared. Furthermore, it is possible to prepare mutants having a modified activity-temperature-profile.

Furthermore, in the case of expression in plants, the insertion of mutations into a nucleic acid molecule of the invention allows the gene expression rate and/or the activity of the proteins encoded by the nucleic acid molecules of the invention to be increased.

For genetic engineering in prokaryotic cells, a nucleic acid molecule of the invention or parts of such a molecule can be introduced into plasmids which permit mutagenesis or sequence modification by recombination of DNA sequences. Standard methods (see Sambrook et al., 1989, Molecular Cloning: A laboratory Manual, 2^{nd} edition, Cold Spring Harbor Laboratory Press, NY, USA) allow base exchanges to be performed or natural or synthetic sequences to be added. DNA fragments can be connected to each other by applying adapters and linkers to the fragments. Moreover, engineering measures which provide suitable restriction sites or remove surplus DNA or restriction sites can be used. In those cases, in which insertions, deletions or substitutions are possible, in vitro mutagenesis, “primer repair”, restriction or ligation can be used. In general, a sequence analysis, restriction analysis and other methods of biochemistry and molecular biology are carried out as analysis methods.

Another embodiment of the invention relates to host cells, in particular prokaryotic or eukaryotic cells transformed with an above-described nucleic acid molecule of the invention or with a vector of the invention, and to cells descended from such transformed cells and containing a nucleic acid molecule or vector of the invention. According to another preferred embodiment, the host cells are cells of microorganisms. In the context of the present invention, the term “microorganism” comprises bacteria and all protists (e.g. fungi, in particular yeasts, algae) as defined Schlegel's “Allgemeine Mikrobiologie” (Georg Thieme Verlag, 1985, 1-2). A preferred embodiment of the invention relates to cells of algae and host cells belonging to the genera Aspergillus, Bacillus, Saccharomyces or Pichia (Rodriguez, Journal of Biotechnology 33 (1994), 135-146, Romanos, Vaccine, Vol 9 (1991), 901 et seq.). A particularly preferred embodiment of the invention relates to E. coli cells. The preparation of such host cells for the production of recombinant proteins can be carried out by methods known to a person skilled in the art.

In a preferred embodiment of the invention, the host cells of the invention show no interfering enzymatic activities, such as those of polysaccharide-forming and/or polysaccharide-degrading enzymes.

An overview of different expression systems is for instance contained in Methods in Enzymology 153 (1987), 385-516, in Bitter et al. (Methods in Enzymology 153 (1987), 516-544) and in Sawers et al. (Applied Microbiology and Biotechnology 46 (1996), 1-9), Billman-Jacobe (Current Opinion in Biotechnology 7 (1996), 500-4), Hockney (Trends in Biotechnology 12 (1994), 456-463), Griffiths et al., Methods in Molecular Biology 75 (1997), 427440). An overview of yeast expression systems is for instance given by Hensing et al. (Antonie van Leuwenhoek 67 (1995), 261-279), Bussineau et al. (Developments in Biological Standardization 83 (1994), 13-19), Gellissen et al. (Antonie van Leuwenhoek 62 (1992), 79-93, Fleer (Current Opinion in Biotechnology 3 (1992), 486-496), Vedvick (Current Opinion in Biotechnology 2 (1991), 742-745) and Buckholz (Bio/Technology 9 (1991), 1067-1072).

Expression vectors have been widely described in the literature. As a rule, they contain not only a selection marker gene and a replication-origin ensuring replication in the host selected, but also a bacterial or viral promoter, and in most cases a termination signal for transcription. Between the promoter and the termination signal there is at least one restriction site or a polylinker which enables the insertion of a coding DNA sequence. The DNA sequence naturally controlling the transcription of the corresponding gene can be used as the promoter sequence, if it is active in the selected host organism. However, this sequence can also be exchanged for other promoter sequences. It is possible to use promoters producing a constitutive expression of the gene and inducible promoters which permit a deliberate control of the expression of the downstream gene. Bacterial and viral promoter sequences possessing these properties are described in detail in the literature. Regulatory sequences for the expression in microorganisms (for instance E. coli, S. cerevisiae) are sufficiently described in the literature. Promoters permitting a particularly high expression of the downstream gene are or instance the T7 promoter (Studier et al., Methods in Enzymology 185 (1990), 60-89), lacUV5, trp, trp-lacUV5 (DeBoer et al., in Rodriguez and Chamberlin (Eds), Promoters, Structure and Function; Praeger, N.Y., (1982), 462-481; DeBoer et al., Proc. Natl. Acad. Sci. USA (1983), 21-25), Ip1, rac (Boros et al., Gene 42 (1986), 97-100). As a rule, the protein amounts are highest from the middle up to about the end of the logarithmic phase of the growth cycle of the microorganisms. Therefore, inducible promoters are preferably used for the synthesis of proteins. These promoters often lead to higher protein yields than do constitutive promoters. The use of highly constitutive promoters leads to the continuous transcription and translation of a cloned gene and, thus, often has the result that energy is lost for other essential cells functions with the effect that cell growth is slowed down (Bernard R. Glick/Jack J. Pasternak, Maolekulare Biotechnologie (1995). Spektrum Akademischer Verlag GmbH, Heidelberg, Berlin, Oxford, p. 342). Therefore, in order to obtain an optimum amount of protein, a two-stage process is often used. First, the host cells are cultured under optimum conditions up to a relatively high cell density. In the second step, transcription is then induced depending on the type of promoter used. In this connection, a tac promoter is particularly suitable which can be induced by lactose or IPTG (=isopropyl-β-D-thiogalactopyranoside) (deBoer et al., Proc. Natl. Acad. Sci. USA 80 (1983), 21-25). Termination signals for transcription are also described in the literature.

The transformation of the host cell with DNA encoding a protein involved in starch degradation can, as a rule, be carried out by standard methods, as for instance described in Sambrook et al., (Molecular Cloning: A Laboratory Course Manual, 2^{nd} edition (1989) Cold Spring Harbor Press, New York; Methods in Yeast Genetics, A Laboratory Course Manual, Cold Spring Harbor Laboratory Press, 1990). The host cell is cultured in nutrient media meeting the requirements of the particular host cell used, in particular in respect of the pH value, temperature, salt concentration, aeration, antibiotics, vitamins, trace elements etc.

Moreover, the invention relates to proteins and biologically active fragments thereof, which are encoded by a nucleic acid molecule of the invention and to methods for their preparation, wherein a host cell according to the invention is cultured under conditions permitting the synthesis of the protein, and the protein is subsequently isolated from the cultured cells and/or the culture medium.

According to a preferred embodiment the protein of the invention is a recombinantly produced protein. In the context of the present invention this is a protein prepared by inserting a DNA sequence encoding the protein into a host cell and expressing it therein. The protein can then be isolated from the host cell and/or the culture medium.

The nucleic acid molecules of the invention now allow host cells to be prepared which produce recombinant proteins of the invention of high purity and/or in sufficient amounts. Within the framework of the present invention the term “high purity” means that the protein according to the invention displays a degree of purity of at least 80%, preferably of at least 90%, even more preferably of at least 95%.

The protein produced by the host cells can be purified by conventional purification methods, such as precipitation, ion exchange chromatography, affinity-chromatography, gel filtration, HPLC Reverse Phase Chromatography etc.

The modification of the nucleic acid molecules of the invention expressed in the host cells, allows to produce a polypeptide in the host cell which is easier to isolate from the culture medium because of particular properties. Thus, the protein to be expressed can be expressed as a fusion protein with an additional polypeptide sequence, the specific binding properties of which permit the isolation of the fusion protein by affinity chromatography (e.g. Hopp et al., Bio/Technology 6 (1988), 1204-1210; Sassenfeld, Trends Biotechnol. 8 (1990), 88-93).

Furthermore, the present invention also relates to an antibody specifically recognizing a protein according to the invention. The antibody can be monoclonal or polyclonal and can be prepared according to methods well known in the art. The term “antibody” also comprises fragments of an antibody which still retain the binding specificity.

The provision of the nucleic acid molecules of the invention makes it possible to prepare plant cells containing and expressing a nucleic acid molecule of the invention by means of genetic engineering.

The invention, therefore, also relates to transgenic plant cells transformed by a nucleic acid molecule of the invention or a vector of the invention or descended from such cells, the nucleic acid molecule being under the control of regulatory elements permitting the transcription of a translatable mRNA in plant cells.

The introduction of the activity of the proteins of the invention, for instance by expression of corresponding nucleic acid molecules, opens the possibility of producing plant cells with an increased starch degradation. Hence, by the expression of a nucleic acid molecule of the invention in plant cells it is possible to express a starch degrading activity which was previously not present in the wild type cell or to increase a starch degrading activity, which was already present in the wild-type cells, by an additional expression. In this regard it might in particular be interesting to increase the starch degrading activity in the endosperm of cereals in particular of cereals used in the production of alcohol, such as beer. An example, is in particular barley. Moreover, it is possible to modify the nucleic acid molecules of the invention according to methods known to a skilled person, in order to obtain enzymes of the invention which for instance possess modified temperature dependencies or substrate or product specificities. Such methods have already been described in more detail in a different context above.

A plurality of techniques is available by which DNA can be inserted into a plant host cell. These techniques include the transformation of plant cells by T-DNA using Agrobacterium tumefaciens or Agrobacterium rhizogenes as a transforming agent, the fusion of protoplasts, injection, electroporation of DNA, insertion of DNA by the biolistic approach and other possibilities.

The use of the Agrobacteria-mediated transformation of plant cells has been extensively investigated and sufficiently described in EP 120 516; Hoekema, In: The Binary Plant Vector System, Offsetdrukkerij Kanters B. V., Alblasserdam (1985), Chapter V; Fraley et al, Crit. Rev. Plant Sci. 4 (1993), 1-46 and An et al., EMBO J. 4 (1985), 277-287. Regarding the transformation of potatoes see for instance Rocha-Sosa et al. (EMBO J. 8 (1989), 29-33).

The transformation of monocotyledonous plants by means of Agrobacterium-based vectors has also been described (Chan et al., Plant Mol. Biol. 22 (1993), 491-506; Hiei et al., Plant J. 6 (1994) 271-282; Deng et al, Science in China 33 (1990), 28-34; Wilmink et al., Plant Cell Reports 11 (1992), 76-80; May et al., Bio/Technology 13 (1995), 486-492; Conner and Dormisse, Int. J. Plant Sci. 153 (1992), 550-555; Ritchie et al. Transgenic Res. 2 (1993), 252-265). An alternative system for transforming monocotyledonous plants is the transformation by the biolistic approach (Van and Lemaux, Plant Physiol. 104 (1994), 37-48; Vasil et al., Bio/Technology 11 (1993), 1553-1558; Ritala et al., Plant Mol. Biol. 24 (1994) 317-325; Spencer et al., Theor. Appl. Genet. 79 (1990), 625-631), protoplast transformation, electroporation of partially permeabilized cells, insertion of DNA via glass fibers. The transformation of maize in particular has been repeatedly described in the literature (see for instance WO 95/06128, EP 0 513 849, EP 0 465 875, EP 29 24 35; Fromm et al, Biotechnology 8, (1990), 833-844; Gordon-Kamm et al., Plant Cell 2, (1990), 603-618; Koziel et al., Biotechnology 11 (1993), 194-200; Moroc et al., Theor. Appl. Genet. 80, (1990), 721-726).

The successful transformation of other types of cereals has also been described for instance of barley (Wan and Lemaux, supra; Ritala et al., supra, Krens et al., Nature 296 (1982), 72-74) and wheat (Nehra et al., Plant J. 5-(1994), 285-297). Generally, any promoter active in plant cells is suitable to express the nucleic acid molecules in plant cells. The promoter can be chosen in a way that the expression in the plants of the invention occurs constitutively or only in a particular tissue, at a particular time of plant development or at a time determined by external influences. The promoter may be homologous or heterologous to the plant.

Suitable promoters are for instance the promoter of 35S RNA of the Cauliflower Mosaic Virus (see for instance U.S. Pat. No. 5,352,605) and the ubiquitin-promoter (see for instance U.S. Pat. No. 5,614,399) which lend themselves to constitutive expression, the patatin gene promoter B33 (Rocha-Sosa et al., EMBO J. 8 (1989), 23-29) which lends itself to a tuber-specific expression in potatoes or a promoter ensuring expression in photosynthetically active tissues only, for instance the ST-LS1 promoter (Stockhaus et al., Proc. Natl. Acad. Sci. USA 84 (1987), 7943-7947; Stockhaus et al., EMBO, J. 8 (1989) 2445-2451), the Ca/b-promoter (see for instance U.S. Pat. No. 5,656,496, U.S. Pat. No. 5,639,952, Bansal et al., Proc. Natl. Acad. Sci. USA 89 (1992), 3654-3658) and the Rubisco SSU promoter (see for instance U.S. Pat. No. 5,034,322; U.S. Pat. No. 4,962,028) or promoters ensuring endosperm specific expression, such as the glutelin promoter from wheat (HMW promoter) (Anderson, Theoretical and Applied Genetics 96, (1998), 558-576, Thomas, Plant Cell 2 (12), (1990), 1171-1180), the glutelin promoter from rice (Takaiwa, Plant Mol. Biol. 30(6) (1996), 1207-1221, Yoshihara, FEBS Lett. 383 (1996), 213-218, Yoshihara, Plant and Cell Physiology 37 (1996), 107-111), the shrunken promoter from maize (Maas, EMBO J. 8 (11) (1990), 3447-3452, Werr, Mol. Gen. Genet. 202(3) (1986), 471-475, Werr, Mol. Gen. Genet. 212(2), (1988), 342-350), the USP promoter, the phaseolin promoter (Sengupta-Gopalan, Proc. Natl. Acad. Sci. USA 82 (1985), 3320-3324, Bustos, Plant Cell 1 (9) (1989), 839-853) or promoters of zein genes from maize (Pedersen et al., Cell 29 (1982), 1015-1026; Quatroccio et al., Plant Mol. Biol. 15 (1990), 81-93). However, promoters which are only activated at a point in time determined by external influences can also be used (see for instance WO 93/07279). In this connection, promoters of heat shock proteins which permit simple induction may be of particular interest. Moreover, seed-specific promoters such as the USP promoter from Vicia faba which ensures a seed-specific expression in Vicia faba and other plants may be used (Fiedler et al., Plant Mol. Biol. 22 (1993), 669-679; Bäumlein et al., Mol. Gen. Genet. 225 (1991), 459-467). Moreover, fruit-specific promoters, such as described in WO 91/01373 may be used too.

Moreover, a termination sequence may be present, which serves to terminate transcription correctly and to add a poly-A-tail to the transcript, which is believed to have a function in the stabilization of the transcripts. Such elements are described in the literature (see for instance Gielen et al., EMBO J. 8 (1989), 23-29) and can be replaced at will.

The transgenic plant cells of the invention can be distinguished from naturally occurring plant cells inter alia by the fact that they contain a nucleic acid molecule of the invention which does either not naturally occur in these cells or, if it does occur naturally in these cells, by the fact that they contain an additional copy or additional copies of such nucleic acid molecules integrated into the genome at sites where it/they naturally does/do not occur. This can be verified, e.g., by Southern blot analysis. Moreover, such transgenic plant cells of the invention can be distinguished from naturally occurring plant cells in that they contain at least one copy of the nucleic acid molecule of the invention stably integrated in their genome.

Moreover, the plant cells of the invention can preferably be distinguished from naturally occurring plant cells by at least one of the following features: If the inserted nucleic acid molecule of the invention is heterologous to the plant cell, then the transgenic plant cells are found to have transcripts of the inserted nucleic acid molecules of the invention. The latter can be detected for instance by Northern blot analysis. The plants cells of the invention preferably contain a protein encoded by an inserted nucleic acid molecule of the invention. This can be shown for instance by immunological methods, in particular by Western blot analysis.

The plant cells according to the invention preferably show an increase in the amount of transcripts from a nucleic acid molecule of the invention of at least 10%, preferably of at least 20%, more preferably of at least 50%, still more preferably of at least 70% and even more preferably of at least 100% when compared to corresponding wild-type plant cells.

Moreover, the plant cells preferably show an increase in the amount of a protein of the invention of at least 10%, preferably of at least 20%, more preferably of at least 50%, still more preferably of at least 70% and even more preferably of at least 100% when compared to corresponding wild-type cells.

In a preferred embodiment the plant cells according to the invention are moreover characterized in that they show an increase of the activity of a protein according to the invention by at least 10%, preferably of at least 20%, more preferably of at least 50%, still more preferably of at least 70% and even more preferably of at least 100% when compared to corresponding wild-type cells. The starch degrading activity can be determined as described above.

Transgenic plant cells can be regenerated to whole plants according to methods known to a person skilled in the art.

The present invention also relates to the plants obtainable by regeneration of the transgenic plant cells of the invention. Furthermore, it relates to plants containing the above-described transgenic plant cells.

The transgenic plants may, in principle, be plants of any plant species, that is to say they may be monocotyledonous and dicotyledonous plants. Preferably, the plants are useful plants cultivated by man for commercial purposes, in particular for nutrition or for technical, in particular industrial, purposes, for example plants which are used in the production of alcohol. They are preferably starch-storing plants, for instance cereal species (rye, barley, cat, wheat, millet, sago etc.), rice, pea, marrow pea, cassava and potato; tomato, rape, soybean, hemp, flax, sunflower, cow pea or arrowroot, fiber-forming plants (e.g. flax, hemp, cotton), oil-storing plants (e.g. rape, sunflower, soybean) and protein-storing plants (e.g. legumes, cereals, soybeans). The invention also relates to fruit plants or trees and palms, e.g. to grapes. Moreover, the invention relates to forage plants (e.g. forage and pasture plants such as grasses, alfalfa, clover, ryegrass) and vegetable plants (e.g. tomato, lettuce, chicory) and ornamental plants (e.g. tulips, hyacinths). Starch-storing plants are preferred. Sugar cane and sugar beet, and potato plants, maize, rice, wheat and tomato plants are particularly preferred.

In the expression of the nucleic acid molecules in plants there exists in principle the possibility that the synthesized protein can be localized in any compartment of the plant cell (e.g. in the cytosol, plastids, vacuole, mitochondria) or the plant (e.g. in the apoplast). In order to achieve the localization in a particular compartment, the coding region must, where necessary, be linked to DNA sequences ensuring localization in the corresponding compartment. The signal sequences used must each be arranged in the same reading frame as the DNA sequence encoding the enzyme.

In order to ensure the localization in the plastids it is conceivable to use one of the following transit peptides: of the plastidic Ferredoxin: NADP+ oxidoreductase (FNR) of spinach which is enclosed in Jansen et al. (Current Genetics 13 (1988), 517-522). In particular, the sequence ranging from the nucleotides −171 to 165 of the cDNA sequence disclosed therein can be used, which comprises the 5′ non-translated region as well as the sequence encoding the transit peptide. Another example is the transit peptide of the waxy protein of maize including the first 34 amino acid residues of the mature waxy protein (Klösgen et al., Mol. Gen. Genet. 217 (1989), 155-161). It is also possible to use this transit peptide without the first 34 amino acids of the mature protein. Furthermore, the signal peptides of the ribulose bisposphate carboxylase small subunit (Wolter et al., Proc. Natl. Acad. Sci. USA 85 (1988), 846-850; Nawrath et al., Proc. Natl. Acad. Sci. USA 91 (1994), 12760-12764), of the NADP malat dehydrogenase (Galiardo et al., Planta 197 (1995), 324-332), of the glutathione reductase (Creissen et al., Plant J. 8 (1995), 167-175) or of the R1 protein Lorberth et al. (Nature Biotechnology 16, (1998), 473-477) can be used.

In order to ensure the localization in the vacuole it is conceivable to use one of the following transit peptides: the N-terminal sequence (146 amino acids) of the patatin protein (Sonnewald et al., Plant J. 1 (1991), 95-106) or the signal sequences described by Matsuoka und Neuhaus, Journal of Experimental Botany 50 (1999), 165-174; Chrispeels und Raikhel, Cell 68 (1992), 613-616; Matsuoka und Nakamura, Proc. Natl. Acad. Sci. USA 88 (1991), 834-838; Bednarek und Raikhel, Plant Cell 3 (1991), 1195-1206; Nakamura und Matsuoka, Plant Phys. 101 (1993), 1-5.

In order to ensure the localization in the mitochondria it is for example conceivable to use the transit peptide described by Braun et al. (EMBO J. 11, (1992), 3219-3227). In order to ensure the localization in the apoplast it is conceivable to use one of the following transit peptides: signal sequence of the proteinase inhibitor II-gene (Keil et al., Nucleic Acid Res. 14 (1986), 5641-5650; von Schaewen et al., EMBO J. 9 (1990), 30-33), of the levansucrase gene from Erwinia amylovora (Geier and Geider, Phys. Mol. Plant Pathol. 42 (1993), 387-404), of a fragment of the patatin gene B33 from Solanum tuberosum, which encodes the first 33 amino acids (Rosahl et al., Mol Gen. Genet. 203-(1986), 214-220) or of the one described by Oshima et al. (Nucleic Acid Res. 18 (1990), 181).

The nucleic acid sequences indicated in SEQ ID NOs: 1, 5 and 7 encode plastidic proteins.

A further subject of the invention is a method for the production of transgenic plant cells and transgenic plants genetically engineered with a nucleic acid molecule of the invention and which in comparison to non-transformed wildtype cells/non-transformed wildtype plants show increased starch degrading activity. In this method the expression and/or the activity of proteins encoded by the nucleic acid molecules of the invention is increased in comparison to corresponding wild-type cells/wildtype plants. In particular, such a method comprises the expression of a nucleic acid molecule according to the invention in plant cells. The nucleic acid molecule according to the invention is preferably linked to a promoter ensuring expression in plant cells. In a particularly preferred embodiment the method comprises the introduction of a nucleic acid molecule according to the invention into a plant cell and regeneration of a plant from this cell.

The increase in expression may, e.g., be detected by Northern blot analysis or Western blot analysis. The increase in activity may be detected by testing protein extracts derived from plant cells for their starch degrading activity. The starch degrading activity can be measured, for instance, as described above or as described in the examples of the present application.

The invention also relates to propagation material of the plants of the invention. The term “propagation material” comprises those components of the plant which are suitable to produce offspring vegetatively or generatively. Suitable means for vegetative propagation are for instance cuttings, callus cultures, rhizomes or tubers. Other propagation material includes for instance fruits, seeds, seedlings, protoplasts, cell cultures etc. The preferred propagation materials are tubers and seeds. The invention also relates to harvestable parts of the plants of the invention such as, for instance, fruits, seeds, tubers or rootstocks.

Furthermore, with the help of the nucleic acid molecules according to the invention it is now also possible to produce plant cells and plants with a reduced activity of a protein according to the invention thereby leading to a reduction of starch degradation. This reduction of the activity may be effected, for example, by means of antisense expression of the nucleic acid molecules of the invention, expression of suitable ribozymes, a cosuppression effect, RNA interference, by the so-called “in vivo mutagenesis”, antibody expression or by the expression of a dominant-negative mutant. Preferably, the reduction of the activity is achieved by inhibiting the expression of an endogenous gene encoding a starch degrading enzyme of the invention.

The term “reduction of starch degradation” preferably means a reduction of the amount of transcripts of at least one nucleic acid molecule of the invention of at least 10%, more preferably of at least 20%, even more preferably of at least 50%, still more preferably of at least 70% and particularly preferred of at least 90% when compared to corresponding wild-type cells.

In another preferred embodiment the term “reduction of starch degradation” means a reduction of the amount of at least one protein of the invention of at least 10%, more preferably of at least 20%, even more preferably of at least 50%, still more preferably of at least 70% and particularly preferred of at least 90% when compared to corresponding wild-type cells.

A “reduction of starch degradation” moreover preferably means a reduction of starch degradation in substantially all cells, organs, tissues or parts of the plants when compared to corresponding wild-type plants or a reduction only in certain cells, organs, tissues or parts of the plants. A reduction most preferably means a reduction of the activity of at least one starch degrading enzyme of the invention of at least 10%, more preferably of at least 20%, even more preferably of at least 50%, in particular of at least 70% and most preferably of at least 90% when compared to corresponding wild-type cells. In a particularly preferred embodiment the starch degradation in the plant cells or plants is completely inhibited. The starch degrading activity of a protein of the invention can be determined as described in the Examples.

In connection with a protein according to the invention which is encoded by SEQ ID NO:7 or a homolog thereof, transgenic plants which show a reduction in the activity of such a protein, preferably to a level of less than about 50% detectable in corresponding wild-type plants, display at least one of the following features:
(i) source leaves, when kept for different time intervals in the dark, have a higher content of starch when compared to leaves of corresponding wild-type plants cultivated under the same conditions (i.e. starch degradation is reduced);
(ii) source leaves of the plants, when growing in the light, have a higher starch content when compared to leaves of corresponding wild-type plants grown under the same conditions, in particular they have a starch content which is at least 150%, more preferably at least 180% and even more preferably at least 240% that of wild-type plants.

In connection with a protein according to the invention which is encoded by SEQ ID NO:5 or a homolog thereof, transgenic plants which show a reduction in the activity of such a protein in comparison to corresponding wild-type plants display at least one of the following features:
(i) source leaves, when kept in the dark for different time intervals, have a higher content of starch in comparison to leaves of corresponding wild-type plants cultivated under the same conditions (i.e. starch degradation is reduced);
(ii) source leaves of the plants, when growing in the light, have a higher starch content when compared to leaves of corresponding wild-type plants grown under the same conditions, in particular they have a starch content which is at least 300%, preferably at least 350%, more preferably at least 400%, still more preferably at least 600%, and particularly preferred at least 800% that of wild-type plants;
(iii) leaves of these plants are longer viable in the dark in comparison to leaves from corresponding wild-type plants.

DNA molecules encoding an antisense RNA which is complementary to transcripts of a DNA molecule of the invention or to sequences of (an) intron(s) of the corresponding genomic sequences are also the subject-matter of the present invention, as well as these antisense molecules. In order to cause an antisense-effect during the transcription in plant cells such DNA molecules have a length of at least 15 bp, preferably a length of more than 100 bp and most preferably a length of more than 500 bp, however, usually less than 5000 bp, preferably shorter than 2500 bp.

The invention further relates to DNA molecules which, during expression in plant cells, lead to the synthesis of an RNA which in the plant cells due to a cosuppression-effect reduces the expression of the nucleic acid molecules of the invention encoding the described protein. Such DNA molecules may comprise the coding region of a nucleic acid molecule of the invention or parts thereof and/or sequences of (an) intron(s) of a corresponding genomic sequence. The invention also relates to RNA molecules encoded thereby. The general principle of cosuppression and the corresponding method is well known to the person skilled in the art and is described, for example, in Jorgensen (Trends Biotechnol. 8 (1990), 340-344), Niebel et al. (Curr. Top. Microbiol. Immunol. 197 (1995), 91-103), Flavell et al. (Curr. Top. Microbiol. Immunol. 197 (1995); 43-56), Palaqui and Vaucheret (Plant. Mol. Biol. 29 (1995), 149-159), Vaucheret et al. (Mol. Gen. Genet. 248 (1995), 311-317) and de Borne e, al. (Mol. Gen. Genet. 243 (1994), 613-621), Smyth (Curr. Biol. 7 (1997), R793-R795) and Taylor, Plant Cell 9 (1997) 1245-1249).

For inhibiting the expression of a nucleic acid molecule according to the invention in plant cells with the help of the above-described antisense approach or with the cosuppression approach, DNA molecules are preferably used which display a degree of homology of at least 90%, more preferably of at least 93%, even more preferably of at least 95% and most preferably of at least 98% with the nucleotide sequence of a corresponding endogenously occurring gene encoding a protein according to the invention.

The invention additionally relates to DNA molecules encoding an RNA which upon expression in a plant cell leads to a reduction of the expression of a nucleic acid molecule of the invention encoding the described protein due to RNA interference (RNAi). The encoded RNA likewise belongs to the scope of the invention.

A similar effect as with antisense techniques can be achieved by producing transgenic plants expressing suitable constructs in order to mediate an RNA interference (RNAi) effect. Thereby, the formation of double-stranded RNA leads to an inhibition of gene expression in a sequence-specific fashion. More specifically, in RNAi constructs, a sense portion comprising the coding region of the gene to be inactivated (or a part thereof, with or without non-translated region) is followed by a corresponding antisense sequence portion. Between both portions, an intron not necessarily originating from the same gene may be inserted. After transcription, RNAi constructs form typical hairpin structures. In accordance with the method of the present invention, the RNAi technique may be carried out as described by Smith (Nature 407 (2000), 319-320) or Marx (Science 288 (2000), 1370-1372).

In a further embodiment the present invention relates to DNA molecules encoding an RNA molecule with ribozyme activity which specifically cleaves transcripts of a DNA molecule of the invention as well as these encoded RNA molecules.

Ribozymes are catalytically active RNA molecules capable of cleaving RNA molecules and specific target sequences. By means of recombinant DNA techniques it is possible to alter the specificity of ribozymes. There are various classes of ribozymes. For practical applications aiming at the specific cleavage of the transcript of a certain gene, use is preferably made of representatives of two different groups of ribozymes. The first group is made up of ribozymes which belong to the group I intron ribozyme type. The second group consists of ribozymes which as a characteristic structural feature exhibit the so-called “hammerhead” motif. The specific recognition of the target RNA molecule may be modified by altering the sequences flanking this motif. By base pairing with sequences in the target molecule these sequences determine the position at which the catalytic reaction and therefore the cleavage of the target molecule takes place. Since the sequence requirements for an efficient cleavage are low, it is in principle possible to develop specific ribozymes for practically each desired RNA molecule.

In order to produce DNA molecules encoding a ribozyme which specifically cleaves transcripts of a DNA molecule of the invention, for example a DNA sequence encoding a catalytic domain of a ribozyme is bilaterally linked with DNA sequences which are homologous to sequences of the target enzyme. Sequences encoding the catalytic domain may for example be the catalytic domains of the satellite DNA of the SCMo virus (Davies et al., Virology 177 (1990), 216-224) or that of the satellite DNA of the TobR virus (Steinecke et al., EMBO J. 11 (1992), 1525-1530; Haseloff and Gerlach, Nature 334 (1988), 585-591). The DNA sequences flanking the catalytic domain are preferably derived from the above-described DNA molecules of the invention. The general principle of the expression of ribozymes and the method is described, for example, in EP-B1 0 321 201. The expression of ribozymes in plant cells is described, e.g., in Feyter et al. (Mol. Gen. Genet. 250 (1996), 329-338).

A reduction of the activity of the protein according to the invention in plant cells can also be achieved by the so-called “in vivo mutagenesis” (also known as “chimeraplasty”). In this method a hybrid RNA/DNA oligonucleotide (chimeroplast) is introduced into cells (Kipp et al., Poster Session at the 5^{th} International Congress of Plant Molecular Biology, Sep. 21 to 27, 1997, Singapore; Dixon and Arntzen, meeting report on “Metabolic Engineering in Transgenic Plants”, Keystone Symposia, Copper Mountain, Colo., USA, TIBTECH 15 (1997), 441-447; international patent application WO 95/15972; Kren et al., Hepatology 25 (1997), 1462-1468; Cole-Strauss et al., Science 273 (1996), 1386-1389; Zhu et al., Proc. Natl. Acad. Sci. USA 96 (1999), 8768-8773). A part of the DNA component of the RNA/DNA oligonucleotide is homologous to a nucleotide sequence occurring endogenously in the plant cell and encoding a protein according to the invention but displays a mutation or comprises a heterologous part which lies within the homologous region. Due to base pairing of the regions of the RNA/DNA oligonucleotide which are homologous to the endogenous sequence with these sequences, followed by homologous recombination, the mutation contained in the DNA component of the oligonucleotide can be introduced into the plant cell genome. This leads to a reduction of the activity of a protein according to the invention.

Furthermore, nucleic acid molecules encoding antibodies specifically recognizing a protein according to the invention in a plant cell, i.e. specific fragments or epitopes of such a protein, can be used for inhibiting the activity of this protein. These antibodies can be monoclonal antibodies, polyclonal antibodies or synthetic antibodies as well as fragments of antibodies, such as Fab, Fv or scFv fragments etc. Monoclonal antibodies can be prepared, for example, by the techniques as originally described in Köhler and Milstein (Nature 256 (1975), 495) and Galfré (Meth. Enzymol. 73 (1981) 3), which comprise the fusion of mouse myeloma cells to spleen cells derived from immunized mammals. Furthermore, antibodies or fragments thereof to the aforementioned peptides can be obtained by using methods which are described, e.g., in Harlow and Lane “Antibodies, A Laboratory Manual”, CSH Press, Cold Spring Harbor, 1988. Expression of antibodies or antibody-like molecules in plants can be achieved by methods well known in the art, for example, full-size antibodies (Düring, Plant. Mol. Biol. 15 (1990), 281-293; Hiatt, Nature 342 (1989), 469-470; Voss, Mol. Breeding 1 (1995), 39-50), Fab-fragments (De Neve, Transgenic Res. 2 (1993), 227-237), scFvs (Owen, Bio/Technology 10 (1992), 790-794; Zimmermann, Mol. Breeding 4 (1998), 369-379; Tavladoraki, Nature 366 (1993), 469-472) and dAbs (Benvenuto, Plant Mol. Biol. 17 (1991), 865-874) have been successfully expressed in tobacco, potato (Schouten, FEBS Lett. 415 (1997), 235-241) or Arabidopsis, reaching expression levels as high as 6.8% of the total protein (Fiedler, Immunotechnology 3 (1997), 205-216).

In addition, nucleic acid molecules encoding a mutant form of a protein according to the invention can be used to interfere with the activity of the wild-type protein. Such a mutant form preferably has lost its starch degrading activity and may be derived from the corresponding wild-type protein by way of amino acid deletion(s), substitution(s), and/or additions in the amino acid sequence of the protein. Mutant forms of such proteins may show, in addition to the loss of kinase activity, an increased substrate affinity and/or an elevated stability in the cell, for instance, due to the incorporation of amino acids that stabilize proteins in the cellular environment. These mutant forms may be naturally occurring or, as preferred, genetically engineered mutants.

Furthermore, it is immediately evident to the person skilled in the art that the above-described antisense, ribozyme, RNA interference, co-suppression, in-vivo mutagenesis, antibody expression and dominant mutant effects can also be used for the reduction of the expression of genes that encode a regulatory protein such as transcription factors, that control the expression of a protein of the invention or, e.g., proteins that are necessary for a protein of the invention to become active.

It is also evident from the disclosure of the present invention that any combination of the above-identified strategies can be used for the generation of transgenic plants, which due to the one or more of the above-described foreign nucleic acid molecules in their cells display a reduced starch degrading activity compared to the corresponding source plant. Such combinations can be made, e.g., by (co-)transformation of corresponding nucleic acid molecules into the plant cell, plant tissue or plant or by crossing transgenic plants that have been generated by different embodiments of the above-described method of the present invention. Likewise, the plants obtainable by the method of the present invention can be crossed with other transgenic plants so as to achieve a combination of increased starch accumulation and another genetically engineered trait, such as for example stress tolerance or a modified starch biosynthesis.

In a further embodiment the present invention relates to vectors containing the above-described DNA molecules the expression of which in a plant cell leads to a reduced activity of a protein of the invention, in particular to vectors in which the described DNA molecules are linked with regulatory elements ensuring the transcription in plant cells.

Furthermore, the present invention relates to host cells containing the described DNA molecules or vectors. The host cell may be a prokaryotic cell, such as a bacterial cell, or a eukaryotic cell. The eukaryotic host cells are preferably plant cells.

Furthermore, the invention relates to transgenic plant cells in which the presence or expression of a foreign nucleic acid molecule leads to the reduction or complete inhibition of the expression of endogenous genes encoding a protein according to the invention.

In a preferred embodiment the foreign nucleic acid molecule is selected from the group consisting of:
(a) DNA molecules encoding an antisense-RNA or an RNAi construct which can lead to a reduction of the expression of endogenous genes encoding a protein according to the invention;
(b) DNA molecules which can lead to a reduction of the expression of endogenous genes encoding a protein according to the invention via a cosuppression-effect;
(c) DNA molecules encoding a ribozyme which can specifically cleave transcripts of endogenous genes encoding a protein according to the invention; and
(d) via in vivo mutagenesis introduced nucleic acid molecules, which lead to a mutation or to an insertion of a heterologous sequence in an endogenous gene encoding a protein according to the invention thereby leading to a reduction of the expression of the protein according to the invention or to the synthesis of an inactive protein.

These transgenic plant cells may be regenerated to whole plants according to well-known techniques. Thus, the invention also relates to plants which may be obtained through regeneration from the described transgenic plant cells, as well as to plants containing the described transgenic plant cells.

Furthermore, the invention relates to the antisense RNA molecules encoded by the described DNA molecules, as well as to RNA molecules with ribozyme activity and RNA molecules which lead to a cosuppression effect or to RNA interference which are obtainable, for example, by means of transcription.

A further subject matter of the invention is a method for the production of transgenic plant cells, which in comparison to non-transformed cells show reduced starch degradation. In this method the amount of protein encoded by a nucleic acid molecule of the invention, which is present in the cells in endogenic form or its activity, is reduced in the plant cells.

In a preferred embodiment this reduction is effected by means of an antisense effect. For this purpose the DNA molecules of the invention or parts thereof are linked in antisense orientation with a promoter ensuring the transcription in plant cells and possibly with a termination signal ensuring the termination of the transcription as well as the polyadenylation of the transcript. Possible is also the use of sequences of (an) intron(s) of corresponding genomic sequences. In order to ensure an efficient antisense effect in the plant cells the synthesized antisense RNA should exhibit a minimum length of 15 nucleotides, preferably of at least 100 nucleotides and most preferably of at least 500 nucleotides. Furthermore, the DNA sequence encoding the antisense RNA should be homologous with respect to the plant species to be transformed.

In a further embodiment the reduction of the amount of proteins encoded by the DNA molecules of the invention is effected by a ribozyme effect. The basic effect of ribozymes as well as the construction of DNA molecules encoding such RNA molecules have already been described above. In order to express an RNA with ribozyme activity in transgenic cells the above described DNA molecules encoding a ribozyme are linked with DNA elements which ensure the transcription in plant cells, particularly with a promoter and a termination signal. The ribozymes synthesized in the plant cells lead to the cleavage of transcripts of DNA molecules of the invention which are present in the plant cells in endogenic form.

A further possibility in order to reduce the amount of proteins encoded by the nucleic acid molecules of the invention is cosuppression. Therefore, the plant cells obtainable by the method of the invention are a further subject matter. These plant cells are characterized in that their amount of proteins encoded by the DNA molecules of the invention is reduced and that in comparison to wild-type cells they show reduced starch degradation.

Likewise, such a reduction of starch degradation may be achieved by exerting an RNA interference effect in the plant cells. For this purpose, a DNA molecule encoding an RNAi construct that is specific for a transcript of a nucleic acid molecule of the invention and that may be prepared according to the working principles of RNA interference explained above may be cloned into a suitable expression vector comprising control elements necessary for expression in plant cells.

Preferably, the transgenic cells show a reduction in the amount of transcripts encoding a protein according to the present invention of at least 10%, more preferably of at least 20%, still more preferably of at least 50%, even more preferably of at least 70% and most preferably of at least 90% in comparison to corresponding non-transformed cells. The amount of transcripts can be determined, for example, by Northern Blot analysis. Furthermore, the cells preferably show a corresponding reduction of the amount of the protein according to the invention. This can be determined, for example, by immunological methods such as Western Blot analysis.

Alternatively, the plant cells show a reduction of the activity of the protein according to the invention of at least 10%, more preferably of at least 20%, still more preferably of at least 50%, even more preferably of at least 70% and most preferably of at least 90% when compared to corresponding non-transformed cells. The activity of a protein of the invention can, e.g., be determined as described in the Examples.

By reducing the amount of the protein of the invention in the cells, the cells show a reduction in starch degradation which may be desirable in certain circumstances as described in the background section. By using suitable promoters the reduction of the starch degrading activity may take place in all or substantially all cells of a plant or may be confined to certain organs, cell types or tissues of the plant, may be induced by external factors or may only take place at a certain developmental stage of the plant

Furthermore, the invention relates to plants obtainable by regeneration of the described plant cells as well as to plants containing the described cells according to the invention.

The transgenic plants may, in principle, be plants of any plant species, that is to say they may be monocotyledonous and dicotyledonous plants. Preferably, the plants are useful plants cultivated by man for commercial purposes, in particular for nutrition or for technical, in particular industrial, purposes, for example plants which are used in the production of alcohol. They are preferably starch-storing plants, for instance cereal species (rye, barley, oat, wheat, millet, sago etc.), rice, pea, marrow pea, cassava and potato; tomato, rape, soybean, hemp, flax, sunflower, cow pea or arrowroot, fiber-forming plants (e.g. flax, hemp, cotton), oil-storing plants (e.g. rape, sunflower, soybean) and protein-storing plants (e.g. legumes, cereals, soybeans). The invention also relates to fruit plants or trees and palms, e.g. to grapes. Moreover, the invention relates to forage plants (e.g. forage and pasture plants such as grasses, alfalfa, clover, ryegrass) and vegetable plants (e.g. tomato, lettuce, chicory) and ornamental plants (e.g. tulips, hyacinths). Starch-storing plants are preferred. Sugar cane and sugar beet, and potato plants, maize, rice, wheat and tomato plants are particularly preferred.

The invention also relates to propagation material of the plants of the invention containing transgenic plants according to the invention. For the definition of the term “propagation material” see above.

Finally, the present invention also relates to the use of a protein according to the invention as an agent for starch degradation in a washing agent or flushing agent as well as to a washing agent or flushing agent comprising a protein according to the invention. Dirtying of clothes are often caused by foodstuff. This foodstuff can contain starch, which is in a state where it sticks together. Washing agents containing starch degradative enzymes should be able to degrade the starch, which is than removed from the dirty fibers. This mechanism supports the cleaning procedure. The same mechanism could support the cleaning of dirt dishes, especially when a dishwasher is used. In a dishwasher the dishes are not cleaned mechanically, in means of scratching the dried foodstuff away from the dishes. Starch degradative enzymes in flushing should also be able to support the cleaning of dishes when a sink is used instead of a dishwasher.

These and other embodiments are disclosed and obvious to a skilled person and embraced by the description and the examples of the present invention. Additional literature regarding one of the above-mentioned methods, means and applications, which can be used within the meaning of the present invention, can be obtained from the state of the art, for instance from public libraries for instance by the use of electronic means. This purpose can be served inter alia by public databases, such as the “medline”, which are accessible via internet, for instance under the address http://www.ncbi.nlm.nih.gov/PubMed/medline.html. Other databases and addresses are known to a skilled person and can be obtained from the internet, for instance under the address http://www.lycos.com. An overview of sources and information regarding patents and patent applications in biotechnology is contained in Berks, TIBTECH 12 (1994), 352-364.

Furthermore, the term “and/or” wherever occurring herein includes the meaning of “and”, “or” and “all or any other combination of elements connected by said term”. All of the above cited disclosures of patents, publications and database entries are specifically incorporated herein by reference in their entirety to the same extent as if each such individual patent, publication or entry were specifically and individually indicated to be incorporated by reference.

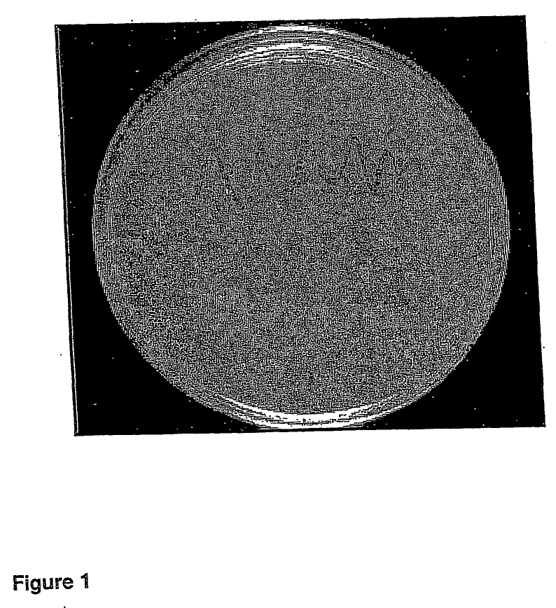

FIG. 1 shows the E. coli strain KV832 expressing the ppt-β-amylase sequence or the empty pSK vector after staining with iodine vapour. Whereas cells containing the empty vector stain blue, the cells expressing the ppt-β-amylase did not stain, indicating that the linear glucans within the cells are degraded by the ppt-β-amylase.

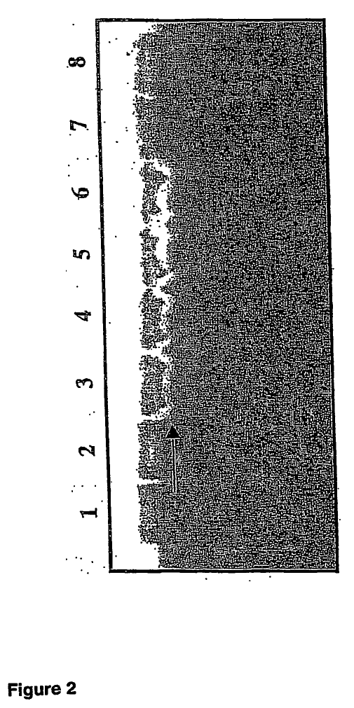

FIG. 2 shows the determination of the hydrolytic activity of the ppt-β-amylase by discontinuous PAGE using separating gels containing amylopectin. As a negative control total soluble protein was separated from E. coli strain BL21-CodonPlus™ RIL expressing the empty pET28a vector (lane 1 and 2 and 7 and 8). In the following four lanes, total soluble protein was separated from E. coli strain BL21-CodonPlus™ RIL expressing the pHIS-ppt-β-amylase (lane 3 to 6). The hydrolytic activity of ppt-β-amylase is detectable by the negative staining of the gels after staining with an iodine solution, indicating that the amylopectin is degraded. The arrow indicates the ppt-β-amylase activity.

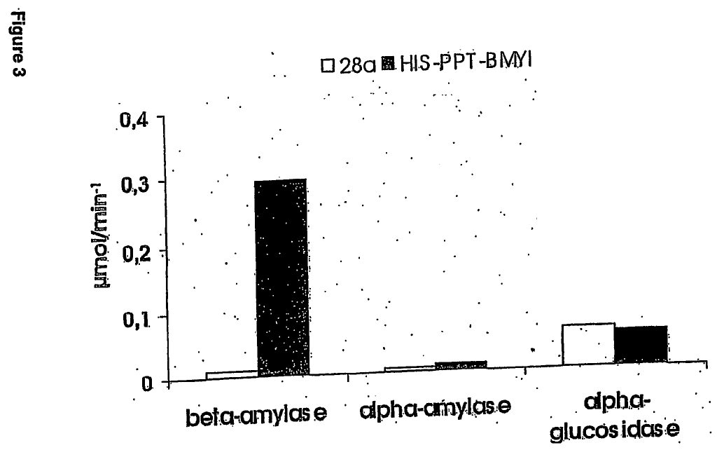

FIG. 3 shows pHIS-ppt-β-amylase activity of the pHIS-BY fusion protein. Hydrolytic activity of E. coli soluble protein fractions from induced cells containing pHIS-ppt-β-amylase fusion protein, with PNPG5 as β-amylase substrate, PNPG7 as α-amylase substrate and p-nitrophenyl-gycoside as a α-glucosidase substrate. As a control the E. coli soluble protein fraction from induced cells containing the empty vector pET28a was used.

FIG. 4 shows the hydrolytic products after incubation of raw and soluble potato starch with the pHIS-ppt-β-amylase fusion protein.

E. coli soluble protein fractions from induced cells containing pHIS-ppt-β-amylase fusion protein and from cells containing the empty vector pET28a were incubated for 12 h with either 10 g L⁻¹ raw or soluble potato starch. The products formed were separated by TLC and stained by charring with sulfuric acid. Standard compounds; G1, Glc; G2-G7, malto-oligosaccarides with chain lengths of two to seven Glc residues. Lane1; pHIS-ppt-β-amylase protein plus raw starch, lane 2; cells containing the empty vector plus raw starch, lane 3; pHIS-ppt-β-amylase protein plus soluble starch, lane 4; containing the empty vector plus soluble starch.

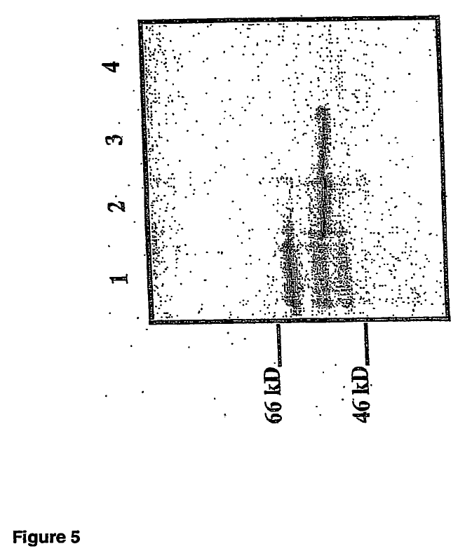

FIG. 5 shows the plastidic targeting of PPT-BMYI.

Pea chloroplasts were incubated with ³⁵S-labeled PPT-BMYI protein. Lane 1 contains the in vitro translated pre-protein used in the import assay. Lane 2 contains the protein isolated from chloroplasts recovered after incubation with the radioactively labelled in vitro translation product. Lane 3 contains protein from chloroplasts incubated with the labelled pre-protein and post-treated with the protease thermolysin. Lane 4 as given for lane 3, except that chloroplasts were post-treated with thermolysin and the detergent Triton X-100. Protein, molecular mass markers in kilodalton are given on the right pre, pre-protein; m, mature protein. Leaves of α-ppt-L-amylase show a starch excess phenotype.

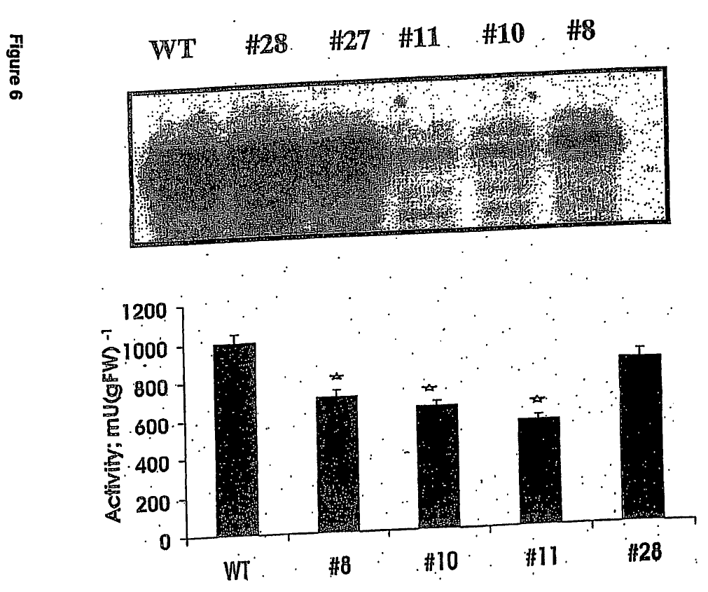

FIG. 6 shows that PPT-BMYI antisense lines display a reduction in the mRNA amount and β-amylase activity.
(a) RNA gel blot analysis of the mRNA amount in source leaves of PPT-BMYI antisense lines stored for one day at 4° C. 20 μg total RNA isolated from different tissues was hybridised with the PPT-BMYI cDNA probe after gel separation and subsequent transfer of the RNA to a nylon membrane.
(b) Determination of β-amylase activity in potato source leaves two hours after the beginning of the dark period. Plant material from source leaves of PPT-BMYI antisense lines were assayed with PNPG5 for β-amylase activity. Results are means±SE of five individual enzyme extracts. * designates a significant difference of the different transgenic lines (#8, #10, #11 and #28) from the corresponding wild type (wt) value at the 5% level (Students t-test).

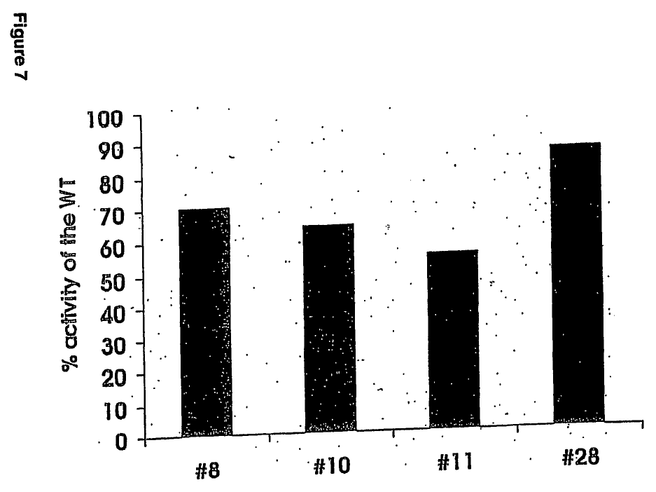

FIG. 7 shows the β-amylase activity in percent in the PPT-BMYI antisense lines in comparison to the untransformed control.

FIG. 8 shows leaves of the x-ppt-β-amylase lines #8, #10, #11, #28 and leaves of the untransformed control which were covered for 72 hours with aluminium foil to keep them in darkness. After this the leaves were stained with an iodine solution for starch. After 72 hours the starch was degraded in wild-type leaves and leaves of line #28, whereas leaves of the lines #8, #10 and #11 still contain starch.

FIG. 9 shows the starch content in 15 weeks old source leaves of the PPT-BMYI antisense lines #8, #10, #11, #28 and the wild-type at the end of the dark/light period. The closed bars represent the starch content at the end of the light period; the open bars the starch content at the end of the light period. Starch content in mmol Hexose equivalents per m²

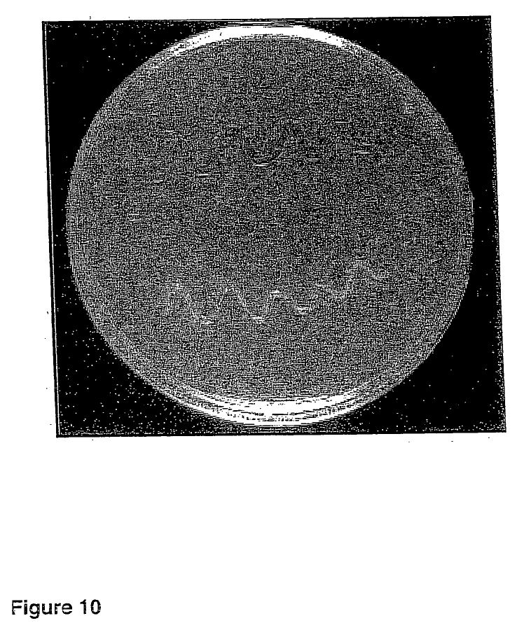

FIG. 10 shows E. coli strain KV832 expressing the CSD23 sequence or the empty pSK vector after staining with iodine vapour. Whereas cells containing the empty vector stain blue, the cells expressing the CSD23 protein did not stain, indicating that the CSD23 protein degrades the linear glucans within the cells.

FIG. 11 shows the determination of the hydrolytic activity of the CSD23 protein by discontinuous PAGE using separating gels containing amylose. As a negative control total soluble protein was separated from E. coli strain DH50α expressing the empty pSK vector (lane 1 to 3). In the following four lanes, total soluble protein was separated from E. coli strain DH5α expressing the CSD23 protein (lane 3 to 7). As a positive control total soluble protein was separated from E. coli strain DH5α expressing a second β-amylase isoform (CF-Beta) of potato (Scheidig, 1987) (lane 8 to 10). The hydrolytic activity of the SHI protein and the β-amylase is detectable by the negative staining of the gels after staining with an iodine solution, indicating that the amylose is degraded.

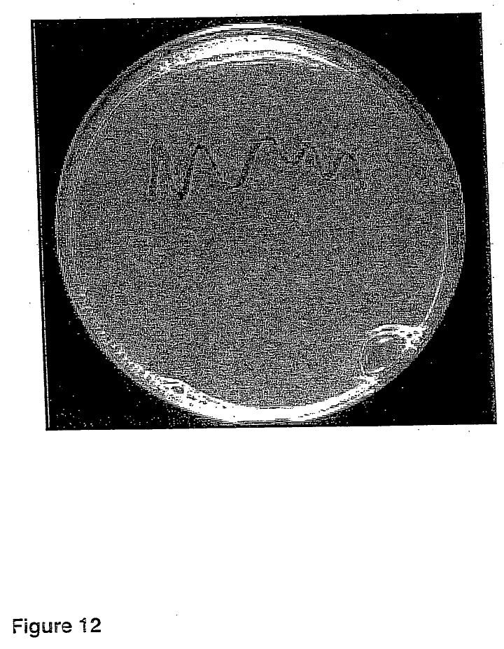

FIG. 12 shows E. coli strain KV832 expressing the CSD12 sequence or the empty pSK vector after staining with iodine vapour. Whereas cells containing the empty vector stain blue, the cells expressing the CSD12 stain lighter blue, indicating that the CSD12 protein degrades the linear glucans within the cells.

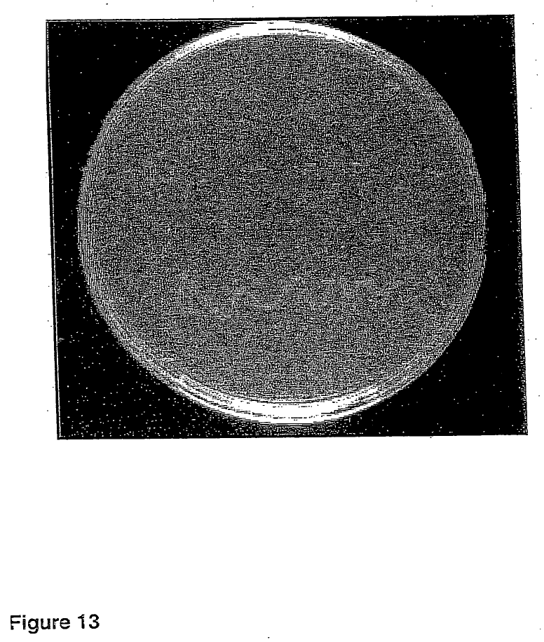

FIG. 13 shows E. coli strain KV832 expressing the SHI sequence or the empty pSK vector after staining with iodine vapour. Whereas cells containing the empty vector stain blue, the cells expressing the SHI protein did not stain, indicating that the SHI protein degrades the linear glucans within them.

FIG. 14 shows the determination of the hydrolytic activity of the SHI protein by discontinuous PAGE using separating gels containing amylopectin. As a negative control total soluble protein was separated from E. coli strain DH5α expressing the empty pSK vector (lane 1 to 3), in the following four lanes, total soluble protein was separated from E. coli strain DH5α expressing the SHI protein (lane 3 to 7). As a positive control total soluble protein was separated from E. coli strain DH5α expressing a second β-amylase isoform (CF-Beta) of potato (Scheidig, 1987) (lane 8 to 10). The hydrolytic activity of the SHI protein and the β-amylase is detectable by the negative staining of the gels after staining with an iodine solution, indicating that the amylopectin is degraded.

FIG. 15 200 μl of total soluble protein of E. coli DH5α cells expressing the SHI protein or the empty pSK vector, were incubated for 24 hours with 200 μl of an amylopectin solution. Afterwards the solutions were stained with iodine to show the degradation of the amylopectin. On the left the negative control (total soluble protein of E. coli DH5α cells expressing the empty vector+amylopectin) on the right total soluble protein of E. coli DH5α cells expressing the SHI protein+amylopectin. Whereas in the negative control the amylopectin stains with iodine, the amylopectin incubated with the SHI protein is degraded and did not stain with iodine.

FIG. 16 shows the hydrolytic products after incubation of soluble potato starch with the SHI protein.

E. coli soluble protein fractions from induced cells containing SHI protein and from cells containing the empty vector pSK was incubated with either 10 g L⁻¹ soluble potato starch. The products formed were separated by TLC and stained by charring with sulfuric acid. Standard compounds; G1, Glc; G2-G7, malto-oligosaccarides with chain lengths of two to seven Glc residues. As a positive control total soluble protein was separated from E. coli strain DH5α expressing a second β-amylase isoform (CF-Beta) of potato and incubated as described with soluble starch (Scheidig, 1987).
1. standard
2., 5. and 8. Soluble protein extract of E. coli DH5α cells expressing the empty vector pSK+soluble starch
3., 6. and 9. Soluble protein extract of E. coli DH5α cells expressing SHI protein+soluble starch
4., 7. and 10. Soluble protein extract of E. coli DH5α cells expressing the CF-Beta protein+soluble starch
11. standard

The incubation time for 2, 3 and 4 was four hours at room temperature, for 5, 6 and 7 six hours and for 8, 9 and 10 eight hours.

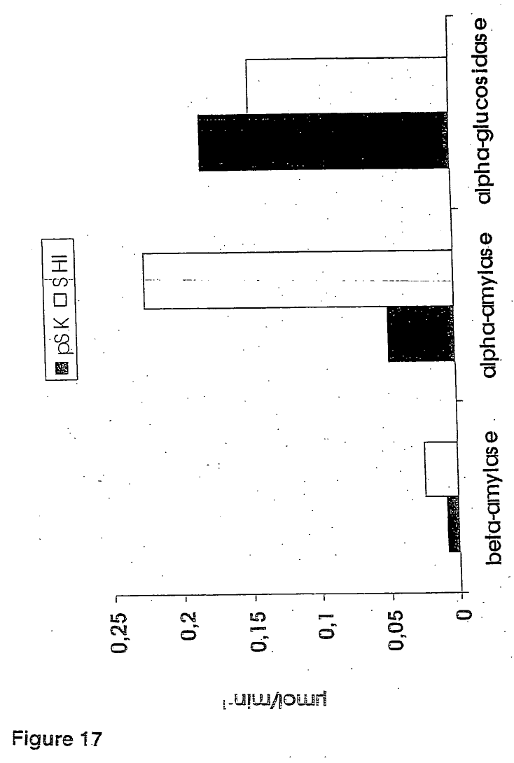

FIG. 17 shows that the SHI protein has α-amylase activity.

Hydrolytic activity of E. coli soluble protein fractions from induced cells containing SHI protein, with PNPG5 as β-amylase substrate, PNPG7 as α-amylase substrate and p-nitrophenyl-gycoside as a α-glucosidase substrate. As a control the E. coli soluble protein fraction from induced cells containing the empty vector pSK was used.

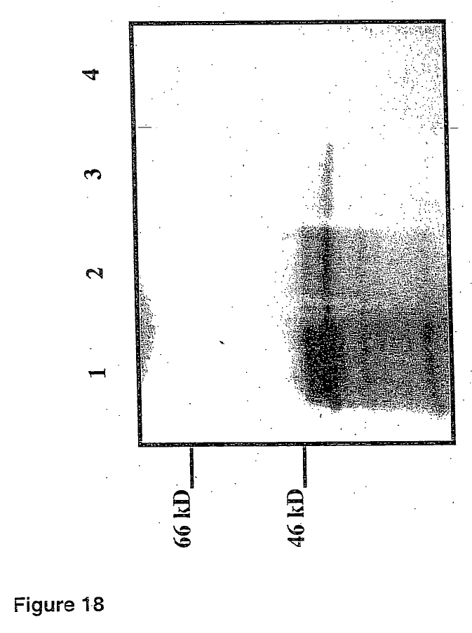

FIG. 18 shows the plastidic targeting of SHI.

Pea chloroplasts were incubated with ³⁵S-labeled SHI100GFP protein. Lane 1 contains the in vitro translated pre-protein used in the import assay. Lane 2 contains the protein isolated from chloroplasts recovered after incubation with the radioactively labelled in vitro translation product. Lane 3 contains protein from chloroplasts incubated with the labelled pre-protein and post-treated with the protease thermolysin. Lane 4 as given for lane 3, except that chloroplasts were post-treated with thermolysin and the detergent Triton X-100. Protein molecular mass markers in kilodalton are given on the right. pre, pre-protein; m, mature protein.

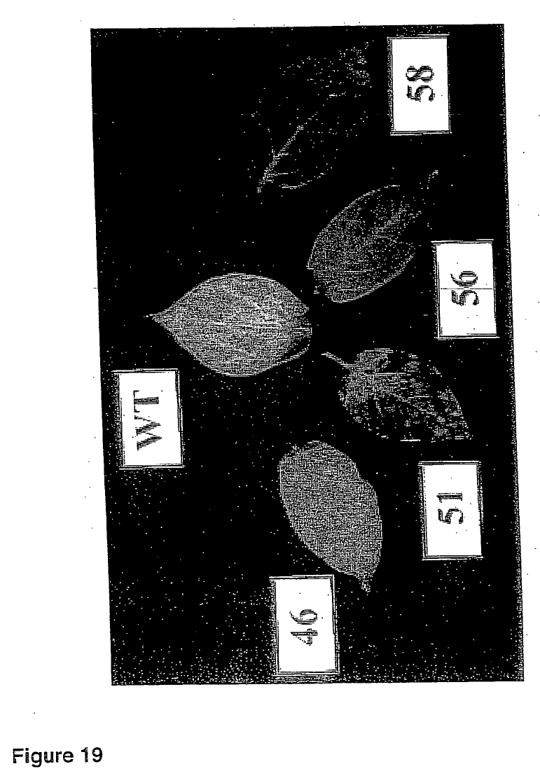

FIG. 19 shows the determination of the degradation of transitory starch in leaves of α-SHI short potato plants. Three lines show a difference in the ability to mobilise the transitory leaf starch in comparison to the wild-type. Leaves of the α-SHI short lines #46, #51, #56, #58 and the wild-type were covered for 64 hours with aluminium foil to keep them in darkness. After this the leaves were stained with an iodine solution for starch. After 64 hours the starch was degraded in wild-type leaves and leaves of line #46, whereas leaves of the lines #51, #56 and #58 still contain starch.

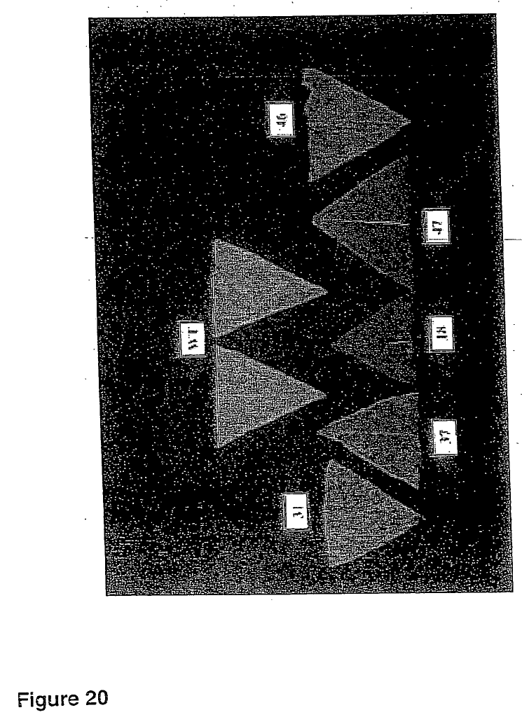

FIG. 20 shows the determination of the degradation of transitory starch in leaves of α-SHI short tobacco plants. Three lines show a difference in the ability to mobilise the transitory leaf starch in comparison to the wild-type. Leaves of the α-SHI short lines #18, #31, #37, #46, #47 and the wild-type were covered for 12 hours with aluminium foil to keep them in darkness. After this the leaves were stained with an iodine solution for starch. After 12 hours the starch was degraded in wild-type leaves whereas leaves of the lines #18, #31, #37, #46, and #47 still contain starch.

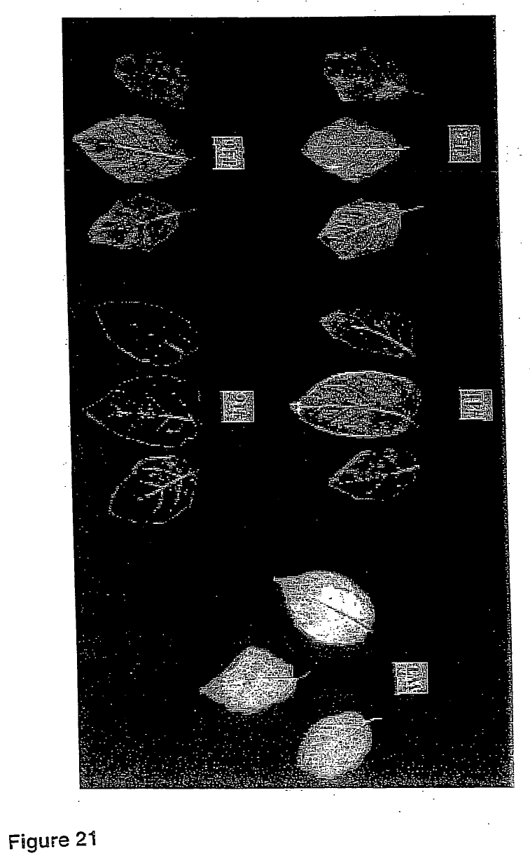

FIG. 21 shows the determination of the degradation of transitory starch in leaves of α-SHIL700 potato plants. Four lines show a difference in the ability to mobilise the transitory leaf starch in comparison to the wild-type. Leaves of the α-SHIL700 lines #16, #41, #46, #53 and the wild-type were covered for 72 hours with aluminium foil to keep them in darkness. After this the leaves were stained with an iodine solution for starch. After 72 hours the starch was degraded in wild-type leaves, whereas leaves of the lines #16, #41, #46, #53 still contain starch.

FIG. 22 shows that leaves of α-SHI short antisense are vital after 14 days in darkness.

Source leaves of SHI short antisense plants (lines #51, #56 and #58) and source leaves of the untransformed control were covered with aluminium foil for 14 days. Whereas leaves of the untransformed are dead, leaves of the antisense lines #51, #56 and #58 (from the left to the right) are still viable.

FIG. 23 shows the starch content in 15 weeks old source leaves of α-SHI short antisense lines #46, #51, #56, #58 and the wild-type at the end of the dark/light period. The open bars represent the starch content at the end of the light period, the closed bars the starch content at the end of the dark period. Starch content in mmol Hexose equivalents per m².

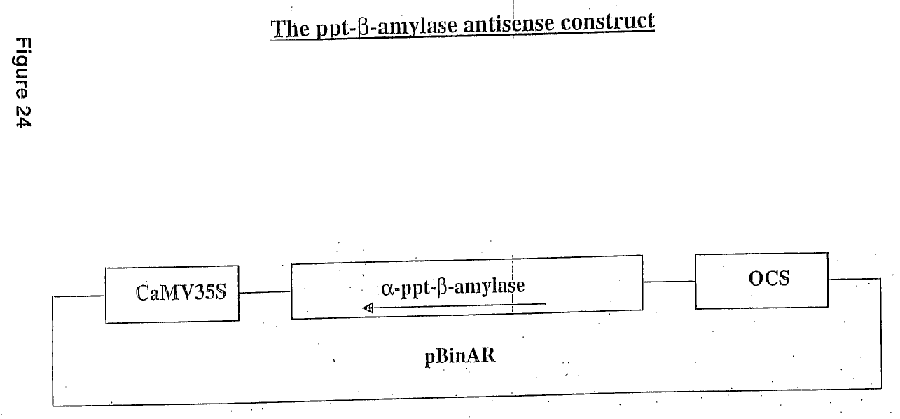

FIG. 24 schematically shows an antisense construct for ppt-β-amylase.

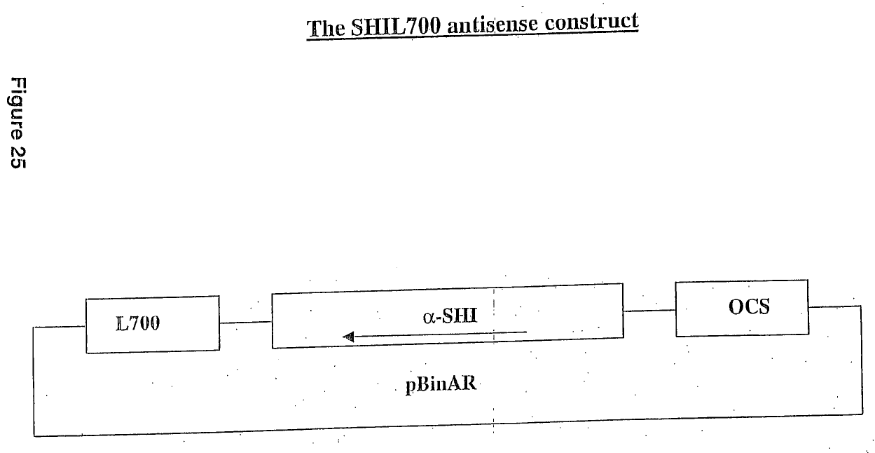

FIG. 25 schematically shows an antisense construct for the SHI protein containing a 2.3 kb fragment of the full-length SHI cDNA.

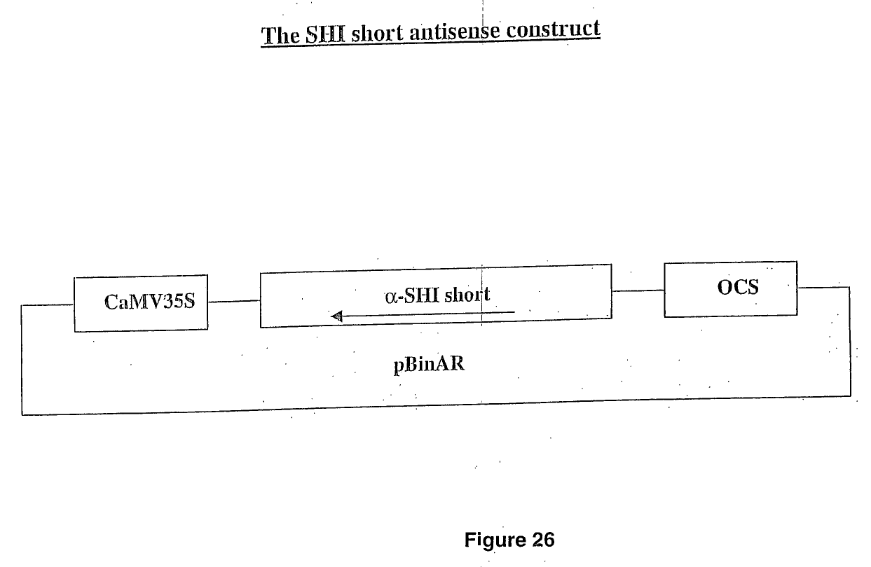

FIG. 26 schematically shows an antisense construct for the SHI protein containing a 1.2 kb fragment of the SHI cDNA.

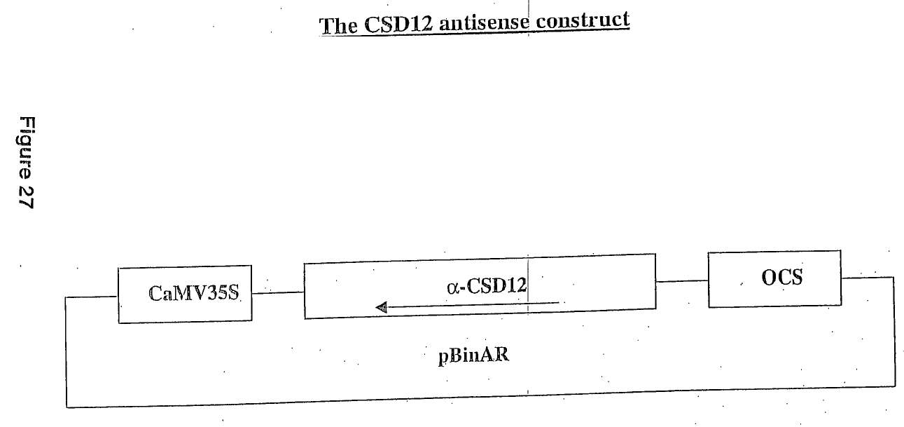

FIG. 27 schematically shows an antisense construct for the CSD12 protein.

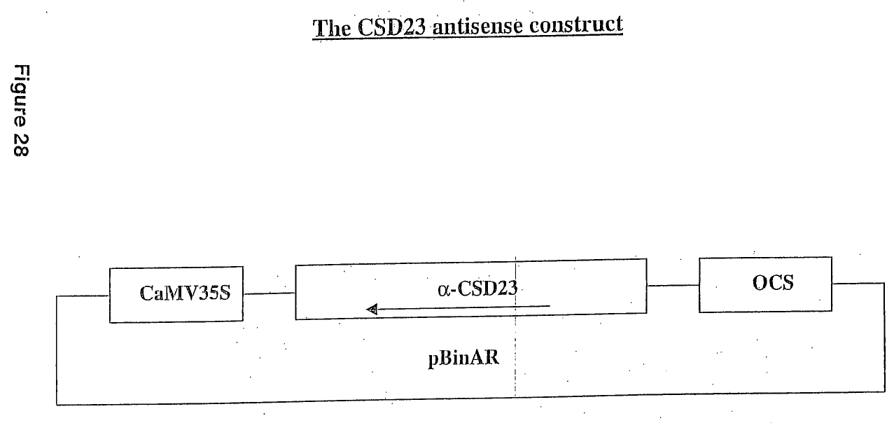

FIG. 28 schematically shows an antisense construct for the CSD23 protein.

The following Examples illustrate the invention.

### EXAMPLE 1

### Cloning of cDNA Coding for Starch Degrading Enzymes

For the isolation of cDNAs coding for starch degrading enzymes, a functional screening approach was used. Two potato λ ZapII cDNA libraries were created. One was made out of potato source-leaf mRNA (leaves harvested every 30 minutes for 2 hours before the light went out and, additionally, 2 hours after), the other was made out of potato tubers, stored for 10 d at 4° C., by using the λ ZapII cDNA synthesis kit (Stratagene). The λ ZapII cDNA libraries were then converted into plasmid libraries by mass in vivo excision according to the manufacturer's protocol.

For functional screening a mutated E. coli strain (KV832) was used which has no glycogen branching enzyme activity. This strain was transformed with the plasmid pACYC-184, (New England Biolabs) containing the E. coli glgC16 gene, that codes for an unregulated form of the enzyme ADP-glucose pyrophosphorylase (Creuzat-Sigal et al., in: Biochemistry of the glycoside linkage, Eds.: Piras and Pontis; New York. USA, Academic Press (1972), 647-680). This plasmid is named pACAG and its construction was described in Kossmann et al. (Planta 208/1999, 503-511). When expressing glgC16, KV832 accumulates large amounts of linear glucans and, because of this, when grown on YT media supplemented with 1% (w/v) glucose, colonies stain blue when exposed to iodine vapour.

KV832 cells containing pACAG were transformed with the plasmid library to obtain 35 000 cfu and grown on solid YT media containing 1% (w/v) glucose, 1 mM IPTG and the appropriate antibiotics at 37° C. overnight. The cells were then stained with iodine vapour. Colonies, which showed either light blue staining or no staining at all (see, e.g., FIG. 1), were isolated and the plasmids within them extracted. The phenotype was confirmed through retransformation of KV832::pACAG. Many plasmids were identified and were separated into different classes following digestion with restriction enzymes. The DNA sequence of the inserts from different plasmids of each class was ascertained using a commercially available service.

### EXAMPLE 2

### Isolation of a cDNA from Potato Encoding a ppt-β-Amylase

Sequence analysis of the insert of one of the plasmids isolated according to Example 1 indicated that it codes for a β-amylase and it was analysed further. The plasmid contains an open reading frame of 1635 bp, coding for a protein with a predicted molecular mass of 61 kD (see SEQ ID NOs: 7 and 8). The sequence was thought to be full length, because a stop codon was found in the 5′ untranslated region immediately before the predicted start codon. This protein shares high amino acid similarity with plant extra-chloroplastidic β-amylases, however, in comparison to these it has an N-terminal extension also. Recently a β-amylase from Arabidopsis was identified also containing an N-terminal extension (Lao et al., Plant J. 20 (1999), 519-527). The authors of this study could show that this β-amylase is targeted to the chloroplast and designated the protein CT-BMY, for chloroplast-targeted β-amylase.

### EXAMPLE 3

### Functional Analysis Shows that ppt-β-Amylase is an Active β-Amylase

To verify whether the ppt-β-amylase described in Example 2 possesses β-amylase activity, the full length cDNA fused to a sequence conferring a His-tag was expressed at high level in E. coli. In order to isolate recombinant ppt-β-amylase protein the ppt-β-amylase cDNA was cloned into the pET expression system. The sequence coding for the ppt-β-amylase protein was amplified by PCR (5′ primer: 5′-gtccgcggatccATGACTTTAACACTTCAATC-3′; lower case nucleotides were added to generate a BamHI side; the T7 primer was used as the 3′ primer), using Pfu-Turbo DNA polymerase (Stratagene). The resulting PCR product was digested with BamHI and Xhol and ligated into the expression vector pET28a (Novagen) to generated pHIS-ppt-β-amylase, which contains a 6×His-Tag at the N-terminus.

Expression of the encoded pHIS-ppt-β-amylase fusion protein was performed at 37° C. in E. coli BL21-CodonPlus™ RIL Competent Cells (Stratagene) with induction by IPTG. Total soluble protein was prepared from induced cells, which contained either the empty expression vector alone, or the vector pHIS-ppt-β-amylase. E. coli cells from 100 ml of cell culture were collected following centrifugation and were re-suspended in 400 μL buffer containing 50 mM Mops-KOH, pH 7.5, 20 mM MgCl₂, 2 mM CaCl₂, 1 mM EDTA, and 0.1% (v/v) β-mercaptoethanol. Approximately 400 μL of glass beads (0.25-0.5 mm diameter) were added and the cells were vortexed four times for 30 sec with pauses on ice to lyse the cells. After centrifugation at 4° C. for 15 min and 20 000 g, the E. coli lysate containing the soluble protein fraction was assayed. Activities for β-amylase and x-amylase were determined by detecting the degradation of malto-oligosaccharides linked to a p-nitrophenyl group by a glucosidic bond at the reducing end, using assay kits from Megazyme (Sydney, Australia). To determine α-glucosidase activity p-nitrophenyl-glucoside was used as a substrate. For the determination of β-amylase activity in E. coli lysates, 50 μL of lysate was mixed with 225 μL of 100 md Mes-KOH, pH 6.2, 1 mM EDTA, and 0.1% (v/v) β-mercaptoethanol. Assays were started by adding 25 μL of substrate and coupling enzyme (final concentration 0.4 mM oligosaccharide and 2.5 units of α-glucosidase) and stopped after 10 min at 40° C., by adding 2.5 volumes of 1% (w/v) Trizma-base (Sigma). The activity was determined as liberated p-nitrophenolate detected spectrophotometrically at 410 nm.

For the determination of β-amylase activity in plant material frozen leaf discs were extracted in 150 μL buffer containing 50 mM Mops-KOH, pH 7.5, 20 mM MgCl₂, 2 mM CaCl₂, 1 mM EDTA, and 0.1% (v/v) β-mercaptoethanol, 3% (w/v) PEG-8000 and 2% (w/v) polyvinylpolypyrrolidone. The samples were centrifuged for 10 min at 4° C. and 20000 g and the supernatant used for measurement of β-amylase activity 25 μL of plant extract were mixed with 250 μL buffer containing 100 mM Mes-KOH, pH 6.2, 1 mM EDTA, and 0.1% (v/v) β-mercaptoethanol. The assay was performed as described above. β-amylase activity was determined by detecting the degradation of malto-oligosaccharides linked to a p-nitrophenyl group by a glucosidic bond at the reducing end. The specific substrate for β-amylase is non-blocked p-nitrophenyl-maltopentaose (PNPG5). The results show that soluble protein fractions from induced cells containing pHIS-ppt-β-amylase reacts with PNPG5, whereas those from control, containing the empty pET28a vector, doesn't. In order to further investigate the catalytic activity, also PNPG7 and p-nitrophenyl-gycoside were used as substrates. PNPG7 is a p-nitrophenyl-maltoheptaose chemically blocked at the non-reducing end, used for the detection of α-amylase activity, p-nitrophenyl-gycoside is a substrate that can be used to determine α-glucosidase activity. The results are shown in FIG. 3. For both substrates no differences between the induced cells containing pHIS-ppt-β-amylase and the empty vector were detectable. This demonstrates that ppt-β-amylase has only β-amylase, and no other amylolytic activity.

It was also possible to show the hydrolytic activity of pHIS-ppt-β-amylase via discontinuous PAGE using separating gels containing amylopectin. For this purpose enzymes were separated by discontinuous PAGE using separating gels containing amylopectin, as described by Hill et al. (Plant Cell Environ. 19 (1996), 1223-1237). Separating gels (0.75 mm) contained 7.5% (w/v) polyacrylamid (30:0.8) acrylamide:bisacrylamide), 0.6% (w/v) potato amylopectin or amylose (Sigma), and 375 mM Tris-HCl (pH 8.8). Stacking gels contained 3% (w/v) polyacrylamid and 63 mM Tris-HCl (pH 6.8). The gels were electrophoresed at 4° C. (Mini Protean 2 system, Bio-Rad) for 1.5 h at a constant current of 30 mA (two gels). After electrophoresis the gels were washed two times in water and incubated for 1.5 h at 20° C. in a buffer containing 0.1 M Mes-KOH (pH 6.2), 2 mM CaCl₂, and 0.1% (v/v) β-mercaptoethanol. Gels were than washed for 10 min in water and stained with iodine to detect degradation of the amylopectin or amylose. The results of these experiments are shown in FIG. 2.

The product of β-amylase activity is maltose only, while α-amylases, for example, produce a series of malto-oligosaccharides. To show that this was the case for pHIS-ppt-β-amylase, the soluble protein fraction was either incubated with solubilised starch or with raw potato starch granules and the products were separated on a TLC-plate. For this purpose 50 μL of E. coli lysate were incubated in buffer containing 100 mM Mes-KOH, pH 6.2, 1 mM EDTA, 0.1% (v/v) β-mercaptoethanol and with either 1% (w/v) soluble, raw potato starch or amylopectin (Sigma). The reaction mixture was applied to a TLC plate (Silicagel F60, Merck). The plate was developed twice with an eluent containing isopropanol:butanol:water (12:3:4). A mixture of glucose and malto-oligosaccharides (two to seven glucose residues) were used as standards. Products formed were visualised by charring of the plates wetted with 10% (v/v) H₂SO₄. Alternatively the reaction mixture was stained with iodine solution for the degradation of soluble starch or amylopectin. The results are shown in FIG. 4. In both cases the hydrolysis product was maltose demonstrating that the pHIS-ppt-β-amylase protein is able to degrade both solubilised starch and potato starch granules.

### EXAMPLE 4

### ppt-β-Amylase is Imported into Isolated Chloroplasts

To determine whether the ppt-β-amylase protein contains a plastid targeting sequence, in vitro protein-import experiments were performed. The ppt-β-amylase cDNA was transcribed in vitro and the product translated in the TNT reticulocyte lysate system according to the instructions of the manufacturer (Promega) using ³⁵S methionine to produce the ³⁵S-labeled pre-ppt-β-amylase. As predicted, the ppt-β-amylase pre-protein had a molecular mass or about 61 kD. Chloroplast were isolated from pea leaves as described by Bartlett et al. (in: Methods in Chloroplasts Molecular Biology; Eds.: Edelmann, Hallick and Chua; Amsterdam, The Netherlands; Elsevier Biochemical Press (1982), 1081-1091). Protein import assays were performed in the light for 30 min at 25° C. in import buffer (250 mM sorbitol, 10 mM methionine, 25 mM potassium gluconate, 2 mM MgSO₄, 50 mM Hepes-KOH, pH 8.0, and 0.2% (w/v) BSA) containing radiolabeled, in vitro-synthesised precursor protein, and purified organelles equivalent to 200 μg of chlorophyll in a final volume of 300 μL.

One fraction was treated with 2.5 μL of thermolysin (1 mg/mL) and 10 μL of 0.1 M CaCl₂ for 20 min on ice. A second fraction contained, additionally, 1% (v/v) Triton X-100. Protease treatment was stopped by the addition of 10 μL of 0.5 M EDTA. Unbroken chloroplasts were re-isolated through a 45% (v/v) Percoll cushion by centrifugation at 4500 g for 8 min, washed in 50 mM Hepes and 0.33 M sorbitol, pH 8.0, and resuspended in 2×SDS sample buffer. The proteins were analysed by electrophoresis (Laemmli, Nature 227 (1970), 680-685) followed by autoradiography. The results are shown in FIG. 5.

When the isolated, intact pea chloroplasts were incubated with ³⁵S-labeled PCT-BMYI in the presence of ATP and appropriate import conditions, nearly all of the added pre-protein was found to be processed into a protein of approximately 55 kD. The addition of the protease thermolysin to the reaction mixture digested the pre-protein in the supernatant, whereas the mature protein that had been imported into the chloroplasts and processed was protected and remained. When a detergent, which destroys the integrity of the plastid membrane and stops the imported protein being protected from proteolysis, was added together with thermolysin all of the labelled protein was digested.

### EXAMPLE 5

### Transgenic Plants with Reduced ppt-β-Amylase Activity Show a Starch-Excess Phenotype

In order to investigate the physiological role of the ppt-β-amylase, especially in respect to transitory starch degradation, transgenic potato plants were generated with reduced ppt-β-amylase activity using an antisense construct (α-ppt-β-amylase); see FIG. 24. For this purpose a 2.1 kb ppt-β-amylase BamHI/Xhol fragment was excised from the pSk vector. The ends of this fragment were filled in using T4 DNA polymerase, to generate blunt ends. This fragment was then cloned in reverse orientation with respect to the Cauliflower Mosaic Virus 35S promoter in the plant expression vector pBinAR via the SmaI restriction side (Höfgen and Willmitzer, Plant Sci. 66 (1990), 221-230) and transformed into potato plants of the variety Désirée using Agrobacterium-mediated gene transfer (Rocha-Sosa et al., EMBO J. 66 (1989), 23-29). Solanum tuberosum L. cv. Désirée was obtained from Saatzucht Fritz Lange KG (Bad Schwartau, Germany). Plants in tissue culture were kept under a 16 h light/8 h dark regime on MS medium (Murashige and Skoog, Physiol. Plant 15 (1962), 473-497) supplemented with 2% (w/v) sucrose at 22° C. Potato plants were transferred from tissue culture and grown in soil under 16 h day and 8 h night regime in a greenhouse. 70 plants were screened for a reduction in ppt-β-amylase mRNA level, in source leaves kept for one day at 4° C. From these 70 plants, three lines (#8, #10, #11) showed a strong reduction in mRNA levels in comparison to the untransformed control, whereas the lines #27 and #28 show no differences in this respect (see FIG. 6a). The lines #8, #10, #11 and #28 were chosen for further investigations. In order to show that a reduction in ppt-β-amylase mRNA level leads to a reduction in ppt-β-amylase activity, PNPG5 was used as a specific β-amylase substrate. The results with PNPG5 show that the β-amylase activity in source leaves of the lines #8, #10, #11, harvested two hours after the light period, is approximately 30-50% of that of the untransformed control, whereas the activity of line #28 was not significantly different from the control (see FIG. 6b and FIG. 7).

In order to evaluate differences in transitory starch degradation, source leaves of 15 weeks old greenhouse grown plants were covered with aluminium foil to keep them in darkness. At different time points leaves were stained for starch content with iodine. In particular, leaves were de-stained in 80% ethanol at 80° C. and subsequently stained with Lugols solution to visualise the starch content. In wild type leaves and those from line #28 the starch was totally degraded after 36 hours, whereas leaves from the lines #8, #10, #11 still contained enough starch so that they stained blue in the presence of iodine. Even after a prolonged time of 72 hours in darkness the lines with the strongest reduction in ppt-β-amylase activity (#10 and #11) contained significant amounts of starch (see FIG. 8).

The starch content in leaves was also determined enzymatically. The leaf starch content in α-ppt-β-amylase plants (lines #8, #10, #11) is much higher in comparison to the untransformed control. At the end of the light period plants of line #8 contained 180%, plants of line #10 and #11 approximately 240%, more starch in their source leaves in comparison to the untransformed control. Starch content was determined as described by Müller-Röber et al. (EMBO J. 11 (1992), 1229-1238). The results are shown in FIG. 9.

### EXAMPLE 6

Isolation of a cDNA (CSD23) from Potato Encoding a Starch Degrading Enzyme

For the isolation of the CSD23 cDNA the procedure as described in Example 1 was used. When this cDNA was expressed in the E. coli strain KV832 under appropriate conditions, the cells do no longer stain blue with iodine vapour (see FIG. 10). Sequence analysis of the CSD23 cDNA insert shows that it codes for a sofar unknown protein. The plasmid contains an open reading frame of 882 bp (see SEQ ID NO: 3), coding for a protein with a predicted molecular mass of 34.1 kD. The sequence was thought to be full length, because a stop codon was found in the 5′ untranslated region immediately before the predicted start codon. This protein shares high amino acid similarity (85.6%) to the unknown protein T05165 of Arabidopsis (Accession No. T05165).

### EXAMPLE 7

### CSD23 Possesses Hydrolytic Activity

In order to verify whether the CSD23 protein possesses hydrolytic activity, the CSD23 protein was expressed in the E. coli strain DH-5α. Expression of the Drotein was performed at 37° C. in E. coli DH5α strain (Bethesda Research Laboratories) with induction by IPTG. Total soluble protein was prepared from induced cells, which contained either the empty pSK vector alone, or the pSK vector containing the sequence encoding the protein. E. coli cells from 100 ml of cell culture were collected following centrifugation and were re-suspended in 400 μL buffer containing 50 mM Mops-KOH, pH 7.5, 20 mM MgCl₂, 2 mM CaCl₂, 1 mM EDTA, and 0.1% (v/v) β-mercaptoethanol. Approximately 400 μL of glass beads (0.25-0.5 mm diameter) were added and the cells vortexed four times for 30 sec with pauses on ice to lyse the cells. After centrifugation at 4° C. for 15 min and 20 000 g, the E. coli lysate containing the soluble protein fraction was assayed. After induction of cell cultures containing the CSD23 sequence in the pSK vector with IPTG, the soluble protein fraction was isolated. As a control the soluble protein fraction was isolated from cells expressing the empty pSK vector alone (isolation of the soluble protein fraction as described for the ppt-β-amylase). The protein fractions were used for the detection of hydrolytic CSD23 activity with discontinuous PAGE using separating gels containing amylose. The results are shown in FIG. 11. In contrast to the soluble protein fraction of the control, the soluble protein fraction of cells expressing CSD23 is able to degrade the amylose in the gels. In addition, separating gels containing amylopectin were used, but no hydrolytic activity was detectable in these gels, indicating that CSD23 only degrades linear and not branched glucans.

For construction of the plant transformation vector α-CSD23, a 1 kb CSD23 XbaI/Asp718 fragment was excised from the pSK vector. The fragment was then cloned in reverse orientation with respect to the Cauliflower Mosaic Virus 35S promoter in the plant expression vector pBinAR (Höfgen and Willmitzer, loc. cit.); see FIG. 28.

Transgenic plants were generated which show a reduction in the CSD23 transcript level.

### EXAMPLE 8

### Isolation of a cDNA (CSD12) from Potato Encoding a Starch Degrading Enzyme

For the isolation of the CSD12 cDNA the procedure as described in Exampe 1 was used. When CSD12 was expressed in the E. coli strain KVS832 under appropriate conditions, the cells do no longer stain blue with iodine vapour (see FIG. 12). The sequence of the CSD12 cDNA is shown in SEQ ID NO: 1.

This CSD12 protein shares high amino acid similarity (83%) to an unknown protein of Arabidopsis (Accession No. AAF01527).

For construction of the plant transformation vector α-CSD12, a 900 bp CSD12 EcoRI/Asp718 fragment was excised from the pSK vector. The fragment was then cloned in reverse orientation with respect to the Cauliflower Mosaic Virus 35S promoter in the plant expression vector pBinAR (Höfgen and Willmitzer, loc. cit.); see FIG. 27.

### EXAMPLE 9

### Isolation of a cDNA (SHI) from Potato Encoding a Starch Degrading Enzyme

For the isolation of the SHI cDNA the procedure as described in Example 1 was used. When SHI was expressed in the E. coli strain KV832 under appropriate conditions, the cells do no longer stain blue with iodine vapour (see FIG. 13). The isolated cDNA did not comprise the full length cDNA. This fragment, designated as SHI short, was used for the construction of the plant transformation vector α-SHI short. The SHI short sequence was then used as a probe to isolate the full length cDNA SHI by standard screening methods. For screening the generated potato leaf λ ZapII cDNA library was used (library described above). The full length SHI cDNA contains an open reading frame of 2370 bp, which encodes a polypeptide of 790 amino acid (see SEQ ID NO: 5). The encoded protein has a predicted molecular mass of 86.6 kD. The predicted protein shares high amino acid similarity (53%) to the unknown Arabidopsis protein F14O13.17 (Accession No. BAB03016).

### EXAMPLE 10

### SHI Possesses Hydrolytic Activity

To verify whether the SHI protein possesses hydrolytic activity the SHI protein was expressed in E. coli strain DH5α. Expression of the protein was performed at 37° C. in E. coli DH5α strain (Bethesda Research Laboratories) with induction by IPTG. Total soluble protein was prepared from induced cells, which contained either the empty pSK vector alone, or the pSK vector containing the sequence encoding the protein. E. coli cells from 100 ml of cell culture were collected following centrifugation and were re-suspended in 400 μL buffer containing 50 mM Mops-KOH, pH 7.5, 20 mM MgCl₂, 2 mM CaCl₂, 1 mM EDTA, and 0.1% (v/v) β-mercaptoethanol. Approximately 400 μL of glass beads (0.25-0.5 mm diameter) were added and the cells vortexed four times for 30 sec with pauses on ice to lyse the cells. After centrifugation at 4° C. for 15 min and 20000 g, the E. coli lysate containing the soluble protein fraction was assayed. The protein fractions were used for the detection of hydrolytic SHI activity. It was possible to show the hydrolytic activity of the SHI protein discontinuous PAGE with separating gels containing amylopectin. The results are shown in FIG. 14. In contrast to the control, the soluble protein fraction of cells expressing SHI is able to degrade the amylopectin in the gels. Furthermore, it could be shown that the recombinant SHI protein is able to degrade solved amylopectin. To show this, an amylopectin solution was incubated with the soluble protein fraction of cells expressing SHI. As a control the protein fraction of cells expressing the empty vector was used. After 6 hours incubation at room temperature the solution was stained with an iodine solution to show the degradation of the amylopectin. The results of this experiment are shown in FIG. 15.

The recombinant SHI protein produces a series of malto-oligosaccharides when the soluble protein fraction is incubated with soluble potato starch (see FIG. 16). This was shown by separating the products on a TLC-plate. The same series of malto-oligosaccharides is produced by α-amylases, indicating that the SHI protein possesses α-amylase activity. Thin layer chromatography was performed as described for the ppt-β-amylase.

### EXAMPLE 11

### SHI Possesses α-Amylase Activity

As described for the ppt-β-amylase described, the hydrolytic activity of the SHI protein was investigated. Then results are shown in FIG. 17. The results show that soluble protein fractions from induced cells containing SHI protein react with PNPG7, whereas those from control, containing the empty pSK vector, do not. In order to further investigate the catalytic activity also PNPG5 and p-nitrophenyl-gycoside were used as substrates. For both substrates no differences between the induced cells containing the SHI protein and the empty vector were detectable. This demonstrates that the SHI has only α-amylase activity, and no other amylolytic activity.

### EXAMPLE 12

### The SHI Protein is Imported into Isolated Chloroplasts

In order to determine whether the SHI protein contains a plastid targeting sequence, in vitro protein-import experiments were performed as described in Example 4. For this experiment a construct was used in which the sequence of SHI coding for the first 100 amino acids of SHI was fused in frame to the green fluorescent protein. When this construct was transcribed and translated in vitro, it was possible to show that the radioactive protein product was imported into chloroplasts (see FIG. 18). These data demonstrate that the first one hundred amino acids of SHI are capable of mediating the transport process.

The import experiments were performed as described above.

### EXAMPLE 13

### Transgenic Plants with Reduced SHI Activity Show a Starch-Excess Phenotype

In order to investigate the physiological role of the SHI protein, especially in respect to transitory starch degradation, transgenic potato and tobacco plants were generated with reduced SHI activity using two different antisense constructs. One construct comprises the SHI short sequence under the control of the 35S-Promoter (α-SHI short). For the construction of this vector, a 1.2 kb SHI BamHI/XhoI fragment was excised from the pSK vector containing the primary isolated SHI sequence. The ends of the fragment were filled in with T4 DNA-Polymerase to generate blunt ends. The fragment was then cloned in reverse orientation with respect to the Cauliflower Mosaic Virus 35S promoter in the plant expression vector pBinAR via the SmaI restriction side (Höfgen and Wilimitzer, loc. cit.); see FIG. 26. The second construct comprises a 2.3 kb fragment of the full length SHI cDNA under the control of the leaf specific L700 promoter (α-SHIL700). For the construction of this vector, a 2.3 kb SHI Asp718/XbaI fragment was excised from the pSK vector containing the full length sequence of SHI. The fragment was then cloned in reverse orientation with respect to the L700 promoter in the plant expression vector pBinAR L700 (Höfgen and Wilimitzer, loc. cit.); see FIG. 25. Both constructs were used for the transformation of potato plant cells as described above. Tobacco was only transformed with the α-SHI short construct.

(a) α-SHI Short Potato Plants

In order to evaluate differences in transitory starch degradation, source leaves of 15 weeks old greenhouse grown plants were covered with aluminium foil to keep them in darkness. At different time points leaves were stained for starch content with iodine. In wild type leaves and those from line #46 the starch was totally degraded after 64 hours in darkness, whereas leaves from the lines #51, #56 and #58 still contained enough starch so that they stained blue in the presence of iodine (see FIG. 19).
The starch content in leaves was also determined enzymatically. The starch content in leaves of α-SHI short plants (lines #51, #56 and #58) is much higher in comparison to the untransformed control. At the end of the light period plants of line #51 contained 412%, plants of line #56 contained 367% and plants of line #58 approximately 814%, more starch in their source leaves in comparison to the untransformed control (see FIG. 23).
   (b) Vitality of Leaves
For leaves of α-SHI short potato plant line # 51, #56 and #58 it could be shown, that leaves of these lines are still vital after a darkperiod of 14 days. In order to show this, leaves were kept in darkness for the indicated time period. Whereas the leaves of the wild-type plants are dead, leaves of the lines # 51, #56 and #58 are still vital. The best result could be shown for leaves of line #58 (see FIG. 22).
   (c) α-SHI Short Tobacco Plants
In order to evaluate differences in transitory starch degradation, source leaves of 10 weeks old greenhouse grown plants were covered with aluminium foil to keep them in darkness. After 12 hours of darkness leaves were stained for starch content with iodine. In wild type leaves the starch was totally degraded after 12 hours in darkness, whereas leaves from the lines #18, #31, #37, #46 and #47 still contained enough starch so that they stained blue in the presence of iodine (see FIG. 20).
   (d) α-SHI L700 Potato Plants
In order to evaluate differences in transitory starch degradation, source leaves of 15 weeks old greenhouse grown plants were covered with aluminium foil to keep them in darkness. At different time points leaves were stained for starch content with iodine. In wild type leaves the starch was totally degraded after 72 hours in darkness, whereas leaves from the lines #16, #41, #46 and #53 still contained enough starch so that they stained blue in the presence of iodine (see FIG. 21).

1. A nucleic acid molecule encoding a protein involved in starch degradation, selected from the group consisting of
(a) nucleic acid molecules encoding at least the mature form of a protein comprising the amino acid sequence indicated in SEQ ID NO: 2;
(b) nucleic acid molecules comprising the nucleotide sequence indicated in SEQ ID NO: 1;
(c) nucleic acid molecules encoding a protein, the amino acid sequence of which has a homology of at least 40% to the amino acid sequence indicated in SEQ ID NO: 2;
(d) nucleic acid molecules the complementary strand of which hybridize with a nucleic acid molecule as defined in (a) or (b);
(e) nucleic acid molecules comprising a nucleotide sequence encoding a biologically active fragment of the protein which is encoded by a nucleic acid molecule as defined in any one of (a), (b), (c) or (d); and
(f) nucleic acid molecules, the nucleotide sequence of which deviates on account of the degeneration of the genetic code from the sequence of a nucleic acid molecule as defined in any one of (b), (c), (d) or (e).

2. A nucleic acid molecule encoding a protein involved in starch degradation, selected from the group consisting of
(a) nucleic acid molecules encoding at least the mature form of a protein comprising the amino acid sequence indicated in SEQ ID NO: 4;
(b) nucleic acid molecules comprising the nucleotide sequence indicated in SEQ ID NO: 3;
(c) nucleic acid molecules encoding a protein, the amino acid sequence of which has a homology of at least 40% to the amino acid sequence indicated in SEQ ID NO: 4;
(d) nucleic acid molecules the complementary strand of which hybridizes with a nucleic acid molecule as defined in (a) or (b);
(e) nucleic acid molecules comprising a nucleotide sequence encoding a biologically active fragment of the protein which is encoded by a nucleic acid molecule as defined in any one of (a), (b), (c) or (d); and
(f) nucleic acid molecules, the nucleotide sequence of which deviates on account of the degeneration of the genetic code from the sequence of a nucleic acid molecule as defined in any one of (b), (c), (d) or (e).

3. A nucleic acid molecule encoding a protein involved in starch degradation, selected from the group consisting of
(a) nucleic acid molecules encoding at least the mature form of a protein comprising the amino acid sequence indicated in SEQ ID NO: 6;
(b) nucleic acid molecules comprising the nucleotide sequence indicated in SEQ ID NO: 5;
(c) nucleic acid molecules encoding a protein, the amino acid sequence of which has a homology of at least 40% to the amino acid sequence indicated in SEQ ID NO: 6;
(d) nucleic acid molecules the complementary strand of which hybridizes with a nucleic acid molecule as defined in (a) or (b);
(e) nucleic acid molecules comprising a nucleotide sequence encoding a biologically active fragment of the protein which is encoded by a nucleic acid molecule as defined in any one of (a), (b), (c) or (d); and
(f) nucleic acid molecules, the nucleotide sequence of which deviates on account of the degeneration of the genetic code from the sequence of a nucleic acid molecule as defined in any one of (b), (c), (d) or (e).

4. A nucleic acid molecule encoding a protein involved in starch degradation, selected from the group consisting of
(a) nucleic acid molecules encoding at least the mature form of a protein comprising the amino acid sequence indicated in SEQ ID NO: 8;
(b) nucleic acid molecules comprising the nucleotide sequence indicated in SEQ ID NO: 7;
(c) nucleic acid molecules encoding a protein, the amino acid sequence of which has a homology of at least 80% to the amino acid sequence indicated in SEQ ID NO: 8;
(d) nucleic acid molecules the complementary strand of which hybridizes under stringent conditions with a nucleic acid molecule as defined in (a) or (b);
(e) nucleic acid molecules comprising a nucleotide sequence encoding a biologically active fragment of the protein which is encoded by a nucleic acid molecule as defined in any one of (a), (b), (c) or (d); and
(f) nucleic acid molecules, the nucleotide sequence of which deviates on account of the degeneration of the genetic code from the sequence of a nucleic acid molecule as defined in any one of (b), (c), (d) or (e).

5. An oligonucleotide or polynucleotide which specifically hybridizes with a nucleic acid molecule of any one of claims 1 to 4.

6. A vector containing a nucleic acid molecule according to any one of claims 1 to 4.

7. The vector according to claim 6, wherein the nucleic acid molecule is connected in sense orientation to regulatory elements ensuring the transcription and synthesis of a translatable RNA in prokaryotic or eukaryotic cells.

8. A host cell transformed with a nucleic acid molecule of any one of claims 1 to 4 or a vector containing said nucleic acid molecule, wherein said nucleic acid molecule is optionally connected in sense orientation to regulatory elements ensuring the transcription and synthesis of a translatable RNA in prokaryotic or eukaryotic cells, or descended from such a cell.

9. he host cell according to claim 8, which is an E. coli cell.

10. The host cell according to claim 9, which is an E. coli cell.

11. A method for preparing a protein involved in starch degradation or a biologically active fragment thereof, wherein a host cell of claim 8 is cultured under conditions permitting the synthesis of the protein, and wherein the protein is isolated from the cultured cells and/or the culture medium.

12. A protein or biologically active fragment thereof encoded by a nucleic acid molecule of claim 1.

13. A transgenic plant cell transformed with a nucleic acid molecule of any one of claims 1 to 4, or descended from such a cell, wherein said nucleic acid molecule encoding the protein involved in starch degradation is under the control of regulatory elements permitting the transcription of a translatable mRNA in plant cells.

14. A plant containing the plant cells of claim 13.

15. The plant according to claim 14, which is a useful plant.

16. The plant according to claim 14, which is a starch-storing plant.

17. The plant of claim 16, which is a potato plant.

18. Propagation material of a plant according to claim 14.

19. A DNA molecule encoding an antisense-RNA complementary to the transcripts of a DNA molecule of any one of claims 1 to 4.

20. A DNA molecule encoding an RNA with ribozyme activity which specifically cleaves transcripts of a DNA molecule of any one of claims 1 to 4.

21. A DNA molecule encoding an RNA which upon expression in a plant cell leads to a reduction of the expression of a nucleic acid molecule of any one of claims 1 to 4 due to a cosuppression effect.

22. A DNA molecule encoding an RNA which upon expression in a plant cell leads to a reduction of the expression of a nucleic acid molecule of any one of claims 1 to 4 due to RNA interference (RNAi).

23. A vector containing a DNA molecule of claim 19.

24. The vector of claim 23, wherein the DNA molecule is combined with regulatory DNA elements ensuring transcription in plant cells.

25. A host cell containing a DNA molecule of claim 19.

26. A transgenic plant cell in which the presence or expression of a foreign nucleic acid molecule leads to a reduced endogenous acitivity of a protein of claim 12.

27. A transgenic plant cell in which the presence or expression of a foreign nucleic acid molecule lends to a reduced endogenous acitivity of a protein of claim 12, wherein said reduced endogenous activity is due to the inhibition of the expression of an endogenous gene encoding a protein of claim 12.

28. A transgenic plant cell in which the presence or expression of a foreign nuclic acid molecule leads to a reduced endogenous activity of a protein due to the inhibition of the expression of an endogenous gene encoding a protein according to claim 12, wherein the foreign nucleic acid molecule is selected from the group consisting of:
(a) DNA molecules encoding an antisense-RNA or an RNAi construct which can lead to a reduction of the expression of endogenous genes encoding a protein of claim 12;
(b) DNA molecules which can lead to a reduction of the expression of endogenous genes encoding a protein of claim 12 via a cosuppression-effect;
(c) DNA molecules encoding a ribozyme which can specifically cleave transcripts of endogenous genes encoding a protein of claim 12; and
(d) via in vivo mutagenesis introduced nucleic acid molecules, which lead to a mutation or to an insertion of a heterologous sequence in an endogenous gene encoding a protein of claim 12 thereby leading to a reduction of the expression of the protein of claim 12 or to the synthesis of an inactive protein.

29. A transgenic plant containing a plant cell of any one of claims 26 to 28.

30. An RNA molecule obtainable by transcription of a DNA molecule of claim 19.

31. A method for the production of transgenic plant cells showing reduced starch degradation characterized in that the amount of a protein of claim 12, which is synthesized in the cells in endogenous form, is reduced in the cells compared to corresponding wild-type cells.

32. The method of claim 31 characterized in that the reduction of the amount of said protein in the cells is caused by an antisense effect.

33. The method of claim 31 characterized in that the reduction of the amount of said protein in the cells is caused by a ribozyme effect.

34. The method of claim 31 characterized in that the reduction of the amount of said protein in the cells is caused by a cosuppression effect.

35. The method of claim 31 characterized in that the reduction of the amount of said protein in the cells is caused by a mutation in the endogenous gene(s) encoding this protein, said mutation being introduced via in vivo mutagenesis.

36. The method of claim 31 characterized in that the reduction of the amount of said proteins in the cells is caused by an RNA interference (RNAi) effect.

37. A plant cell obtainable by a method of any one of claims 31 to 36.

38. A transgenic plant containing plant cells of claim 37.

39. Propagation material of a transgenic plant containing plant cells of any one of claims 26 to 28.

40. A washing or flushing agent comprising a protein of claim 12.

41. Propagation material of a transgenic plant containing plant cells of claim 37.
